(19) United States
(12) Patent Application Publication
(10) Pub. No.: US 2025/0288549 A1
(43) Pub. Date: Sep. 18, 2025

(54) SYNTHETIC PROCESS FOR PRODUCTION OF LIPOXIN B4 AND ANALOGUES THEREOF

(71) Applicant: UNIVERSITY HEALTH NETWORK, Toronto (CA)

(72) Inventors: Mark Andrew REED, Puslinch (CA); Carla BROWN, Toronto (CA); Chien-Hsun Frank LEE, Mount Albert (CA); Alexander James NIELSEN, Hamilton (CA); Jeremy SIVAK, Toronto (CA)

(21) Appl. No.: 18/860,997

(22) PCT Filed: May 1, 2023
(86) PCT No.: PCT/CA2023/050587

§ 371 Date: Oct. 28, 2024

Related U.S. Application Data
(60) Provisional application No. 63/336,485, filed on Apr. 29, 2022.

(51) Int. Cl. A61K 31/202; A61P 25/28; C07C 51/09; C07C 59/42

(52) U.S. Cl. CPC A61K 31/202; A61P 25/28; C07C 51/09; C07C 59/42; C07B 2200/05

(57) ABSTRACT

The present application provides a synthetic process for production of lipoxin B4 and analogues thereof, which is modular and scalable, thus permitting synthesis of large quantities of LXB₄ and analogues thereof, including radiolabeled analogues. Also provided are intermediate compounds that are useful in the synthetic process for production of lipoxin B4 and analogues thereof.

### CROSS-REFERENCE TO RELATED APPLICATION

This application claims priority to U.S. Provisional Patent Application No. 63/336,485, filed on Apr. 29, 2022, the contents of which is incorporated herein by reference in its entirety.

### FIELD OF THE INVENTION

The present application pertains to the field of lipoxin synthesis. More particularly, the present application relates to a synthetic process for producing lipoxin B4 and analogues thereof, as well as intermediate compounds useful in the synthesis of lipoxin B4 and analogues thereof.

### INTRODUCTION

Lipoxin B4 (LXB₄) is a non-classic eicosanoid and a member of the specialized pro-resolving mediator (SPM) family of polyunsaturated fatty acids, naturally derived from arachidonic acid (AA) through a series of oxidation steps by 5-lipoxygenase (5-LOX) and 12/15-LOX.[1,2] Extensive chromatographic evidence along with comparisons of biological activities to related eicosanoids led to the structural and configurational assignment of LXB₄ [3,4] and related lipoxin A4 (LXA₄) (FIG. 1).[5] Both lipoxins have been associated with the promotion of anti-inflammatory and pro-resolution processes since their initial discovery in 1984.[6] Of the two structurally similar lipoxins, LXA4 has been well-studied and shown to primarily bind to the G-protein coupled formyl peptide receptor 2 (ALX/FPR2) and GPR32, which initiates downstream anti-inflammatory effects.[7,8] However, endogenous receptors for LXB₄ have so far not been identified. Additionally, spectroscopic analysis to confirm the structure of LXB₄ remains ambiguous, even though it has been the subject of previous total syntheses and is reported to exhibit potent bioactivities.[9-11]

The first total synthesis of LXB₄ was reported in 1985 and employed a chiral pool strategy from 2-deoxy-D-ribose.[12a] LXB₄ and its all-trans isomer were reported in order to assign the stereochemistry relative to an authentic natural sample. Shortly thereafter in 1986, a very similar strategy was utilized to synthesize LXB₄ and its methyl ester for the same purpose.[12b] Also in 1986, a stereocontrolled total synthesis of LXB₄ and its isomers was reported, which used Sharpless asymmetric epoxidation and asymmetric reduction strategies to install the stereocentres in the natural product.[12c] Since then, other formal and total syntheses of LXB₄ have been reported.[12d-f] However, these previously reported syntheses are all generally low yielding, have long synthetic routes, and lack full spectral characterization for LXB₄ product.

Recently, both LXA₄ and LXB₄ have been implicated in the regulation of neuroinflammation and neurodegeneration.[13] Specifically, therapeutic LXB₄ treatment was found to be significantly more potent and efficacious than LXA₄ in promoting direct neuroprotection in a variety of neuronal cell types, and from acute and chronic injury models of the neurodegenerative disease glaucoma.[14] International PCT Patent Application WO 2018/161175, which is incorporated herein by reference in its entirety, teaches that LXB₄, and compositions that comprise LXB₄, is useful for inhibiting or preventing neurodegeneration, specifically hippocampal, cortical, and/or retinal ganglion cell neurons (RGC), and degeneration and/or cell loss or treating related disorders and diseases using lipoxin.

Consequently, and given the drawbacks associated with previous syntheses, there is a need for an efficient synthesis of LXB₄ and analogues or derivatives thereof, to facilitate drug discovery programs to study LXB₄ signaling and to enable the identification and deconvolution of LXB₄'s putative biological targets.

The above information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present application is to provide a synthetic process for production of lipoxin B4 and analogues thereof. This object is met by the presently described modular and scalable synthetic approach, which permits synthesis of large quantities of LXB₄, thereby facilitating study of its role in neuronal signaling mechanisms and evaluation of its therapeutic potential as a treatment for neurodegeneration. Further, the present inventors have, for the first time, comprehensively characterized the structure of LXB₄ using a combination of 1D and 2D ¹H and ¹³C NMR, high resolution mass spectrometry and optical rotation, confirming that the structure of LXB₄ is indeed (5S,6E,8Z,10E,12E,14R,15S)-5,14,15-trihydroxyicosa-6,8,10,12-tetraenoic acid (2), as shown in FIG. 1.

In accordance with one aspect, there is provided a process for synthesizing a compound of Formula I,

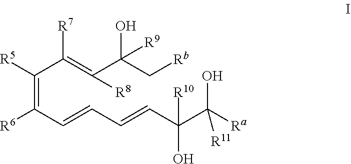

wherein
R⁵—R¹¹ are each independently H, ²H or ³H, optionally where at least one of R⁵—R¹¹ is ²H or ³H,
R^{a} is an optionally substituted alkyl or alkylene, and
R^{b} is an optionally substituted alkyl or alkylene,
   said process comprising the steps of:
a) reacting a compound of Formula III with a vinyl halide of Formula IV to produce a compound of Formula V

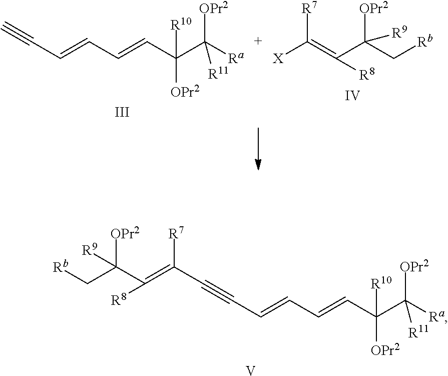

wherein each Pr² is independently an alcohol protecting group and X is a halide (e.g., I);
b) deprotecting the compound of Formula V to produce a compound of Formula Va

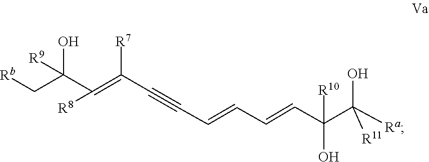

and
c) performing a selective semi-hydrogenation of the triple bond in the compound of Formula Va to form the compound of Formula I.

In some embodiments, the compound of Formula I made by the above process contains one or two deuterium atoms, with the remainder of the R⁵ to R¹¹ being hydrogen. In other embodiments, in the compound of Formula I made by the above process each of R⁵ to R¹¹ is hydrogen. In one example the compound of Formula I made by the above process is LXB₄.

In accordance with another aspect of the present application, there is provided a process for synthesizing a compound of Formula I′,

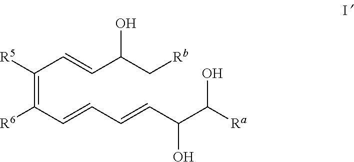

wherein R⁵, R⁶, R^{a} and R^{b} are as defined above,
said process comprising the steps of:
a) reacting a compound of Formula III′ with a vinyl halide of Formula IV′ to produce a compound of Formula V′

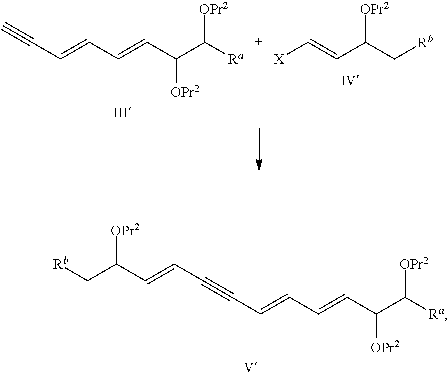

wherein each Pr² is independently an alcohol protecting group and X is a halide (e.g., I);
b) deprotecting the compound of Formula V′ to produce a compound of Formula Va′

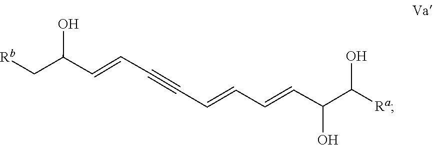

and
c) performing a selective semi-hydrogenation of the triple bond in the compound of Formula Va′ to form the compound of Formula I′.

In one embodiment, the process is for forming compounds of Formula Ia

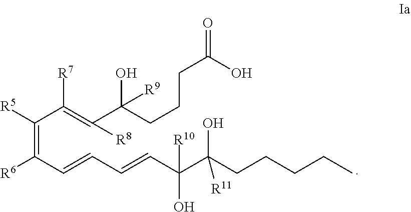

In another embodiment, the process is for forming compounds of Formula Ia′

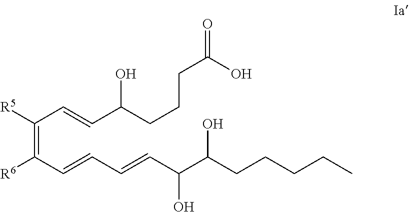

wherein R⁵ and R⁶ are each independently H, ²H or ³H, said process comprising the steps of:
a) treating a compound of Formula III′ with a vinyl iodide of Formula IV′ to produce a Formula V′

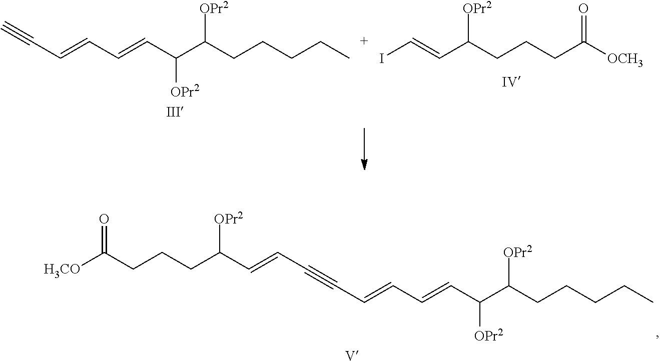

wherein each Pr² is independently an alcohol protecting group;
b) deprotecting the compound of Formula V to produce a compound of Formula Va′

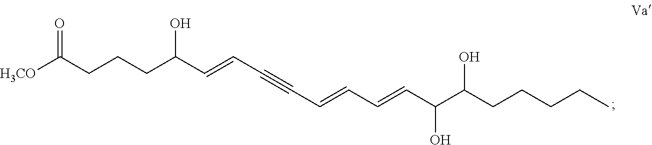 [SMILES: CCCCCC(O)C(O)/C=C/C=C/C#C/C=C/C(O)CCCC(=O)OC]

and
c) performing a selective semi-hydrogenation of the compound of Formula Va′ and subsequent ester hydrolysis to form the compound of Formula Ia′.

In accordance with another aspect, there is provided a compound that has the structure of Formula Ia

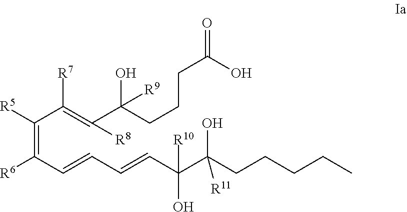

wherein R⁵—R¹¹ are each independently H, ²H or ³H, where at least one of R⁵—R¹¹ is ²H or ³H, or is a pharmaceutically acceptable salt, ester, hydrate, or solvate thereof, optionally wherein the compound has a structure that is:

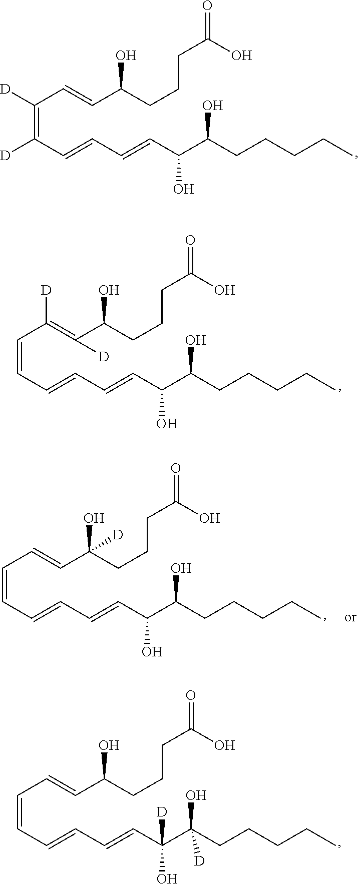

or is a pharmaceutically acceptable salt, ester, hydrate, or solvate thereof.

In accordance with another aspect, there is provided a compound that has the structure of Formula Ia′

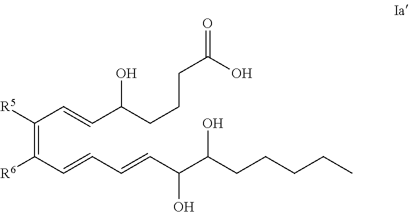

wherein R⁵ and R⁶ are the same and are deuterium or tritium, or that is a pharmaceutically acceptable salt, ester, hydrate, or solvate thereof. When the compound of Formula Ia or Ia′, or the pharmaceutically acceptable salt, ester, hydrate, or solvate thereof, does not include tritium and includes one or two deuterium atoms, it is useful as a neuroprotective agent in treatment or prevention of diseases and/or conditions associated with neuroinflammation or neurodegeneration. Accordingly, another aspect of the present invention provides a method and use for treating or preventing a disease or condition associated with neuroinflammation or neurodegeneration in a subject, comprising administering the compound of Formula Ia or Ia′, which includes at least one deuterium atom, or the pharmaceutically acceptable salt, ester, hydrate, or solvate thereof, to the subject.

In accordance with some embodiments, the deuterium-containing compounds are useful for inhibiting or preventing central nervous system neurodegeneration and/or neural cell loss in the subject in need thereof, optionally wherein the central nervous system neurodegeneration and/or neural cell loss is selected from hippocampal neuron, optic neuron and/or retinal ganglion cell neuron (RGC) degeneration and/or neural cell loss.

In accordance with another aspect, there is provided a compound of Formula II

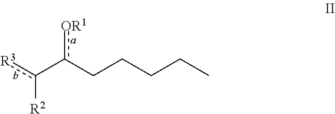

wherein
bond a is a single or double bond;
bond b is a single, double or triple bond;
   when bond a is a single bond, R¹ is H or an alcohol protecting group and when bond a is a double bond, R¹ is absent;
   when bond b is a single bond,
R² is OR′, where R′ is H or an alcohol protecting group, and R³ is —CH═CH—CH═CH≡CH, —CH═CH—CH═O, —CH═CH—CH═CH≡C—Pr¹, where Pr¹ is an alkyne protecting group, —CH(OCH₃)₂, or

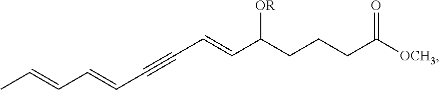

where R is H or an alcohol protecting group, or
R² is O which forms an epoxide with the carbon to which R³ is attached and R³ is —CHO or —CH₂OR″, where R″ is an alcohol protecting group;
   when bond b is a double bond R² is H and R³ is —CH₂OR″, wherein R″ is as defined above; and
   when bond b is a triple bond R² is absent and R³ is —CH₂OR″, wherein R″ is as defined above.

The compound of Formula II is useful, at least, as an intermediate in the production of LXB₄ and analogues thereof.

### BRIEF DESCRIPTION OF TABLES AND FIGURES

For a better understanding of the application as described herein, as well as other aspects and further features thereof, reference is made to the following description which is to be used in conjunction with the accompanying drawings, where:

FIG. 1 depicts the chemical structures of lipoxins A4 (1) and B4 (2); and

FIG. 2 graphically illustrates the results of in vitro validation studies between synthesized LXB₄ vs. commercial LXB₄ from Cayman Chemical. A) HT22 cells were treated with 1 μM of LXB₄ from either a commercial source (Cayman) or synthesized according to the present process. Both showed significant protection in a glutamate injury model (n=8, *p<0.05, bars are S.E.). B) A dose response curve for the LXB₄ synthesized according to the present process in the same assay indicates an EC₅₀ of 292.8 nM (n=8, bars are S.E.).

### DETAILED DESCRIPTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

Reference throughout this specification to “one embodiment,” “an embodiment,” “another embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example of proliferative disorders, including, but not limited to cancer. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “acyl”, as used herein, refers to a carbonyl-containing functionality, e.g., —C(═O)R′, wherein R′ is an aliphatic, alycyclic, heteroaliphatic, heterocyclic, aryl, heteroaryl, (aliphatic)aryl, (heteroaliphatic)aryl, heteroaliphatic(aryl) or heteroaliphatic(heteroaryl) moiety, whereby each of the aliphatic, heteroaliphatic, aryl, or heteroaryl moieties is substituted or unsubstituted, or is a substituted (e.g., hydrogen or aliphatic, heteroaliphatic, aryl, or heteroaryl moieties) oxygen or nitrogen containing functionality (e.g., forming a carboxylic acid, ester, or amide functionality).

As used herein, the term “aliphatic” or “aliphatic group”, refers to a straight chain (i.e., non-branched), branched or cyclic (including fused, bridged and spiro-fused polycyclic) hydrocarbon, which may be fully saturated or contain one or more unsaturated units, but is not aromatic. The term “aliphatic” or “aliphatic group”, as used herein, also includes substituted analogs of these residues in which at least one of the hydrogen atoms of the aliphatic group is replaced by a non-hydrogen substituent. Unless otherwise indicated, aliphatic groups contain 1 to 20 carbon atoms, 1 to 12 carbon atoms, 1 to 8 carbon atoms, 1 to 6 carbon atoms, 1 to 5 carbon atoms, 1 to 4 carbon atoms, 1 to 3 carbon atoms or 1 or 2 carbon atoms. Suitable aliphatic groups include, but are not limited to, straight, branched or cyclic alkyl, alkenyl and alkynyl groups, and hybrids thereof such as (cycloalkyl) alkyl, and (cycloalkenyl) alkyl.

As used herein, the term “alkyl,” unless otherwise specified, refers to a straight or branched chain saturated hydrocarbon radical, for example, methyl, ethyl, n-propyl, isopropyl, n-butyl, sec-butyl, t-butyl, n-pentyl, sec-pentyl, t-pentyl, neopentyl, and the like. In some embodiments, alkyl groups have from 1 to 20 carbon atoms, or from 1 to 12 carbon atoms, or from 1 to 8 carbon atoms, from 1 to 6 carbon atoms, or from 1 to 4 carbon atoms. The term “cycloalkyl” as used herein, is also intended to have its accustomed meaning of a cyclic, saturated hydrocarbon, such as, for example, cyclopropyl, cyclobutyl, cyclopentyl, cyclohexyl, cycloheptyl, cyclooctyl, or the like. In some embodiments, cycloalkyl groups have from 3 to 10 carbon atoms, or from 3 to 8 carbon atoms, or from 3 to 6 carbon atoms, or 5 or 6 carbon atoms.

As used herein, the term “alkenyl,” unless otherwise specified, refers to a monovalent group derived from a straight or branched chain aliphatic moiety having at least one carbon-carbon double bond. Unless otherwise indicated, alkenyl groups contain 2 to 12 carbon atoms. In certain embodiments, the alkenyl group contains 2 to 8 carbon atoms. In certain embodiments, the alkenyl group contains 2 to 6 carbon atoms. In some embodiments, the alkenyl group contains 2 to 5 carbon atoms, and in some embodiments, the alkenyl group contains 2 to 4 carbon atoms, and in another embodiment, the alkenyl group contains 2 to 3 carbons Atoms, and in another embodiment, the alkenyl group contains two carbon atoms. The alkenyl group includes, for example, ethenyl, propenyl, butenyl, butadienyl, 1-methyl-2-buten-1-yl and the like.

As used herein, the term “alkynyl,” unless otherwise specified, refers to a monovalent group derived from a straight or branched chain aliphatic moiety having at least one carbon-carbon triple bond. Unless otherwise indicated, the alkynyl group contains 2 to 12 carbon atoms. In certain embodiments, the alkynyl group contains 2 to 8 carbon atoms. In certain embodiments, the alkynyl group contains 2 to 6 carbon atoms. In some embodiments, the alkynyl group contains 2 to 5 carbon atoms, and in some embodiments, the alkynyl group contains 2 to 4 carbon atoms, and in another embodiment, the alkynyl group contains 2 to 3 carbons Atoms, and in another embodiment, the alkynyl group contains two carbon atoms. Representative alkynyl groups include, but are not limited to, ethynyl, 2-propynyl (propargyl), 1-propynyl, and the like.

As used herein, the term “aryl,” unless otherwise specified, is intended to mean an aromatic hydrocarbon system, for example, phenyl, naphthyl, phenanthrenyl, anthracenyl, pyrenyl, and the like. Included within the term “aryl” are heteroaryl groups including one or more heteroatom in the aromatic system, for example, pyridyl, furyl, and thienyl. In some embodiments, aryl groups have from 6 to 10 carbon atoms. A “substituted aryl” includes one or more substituent, as defined below. Preferably, a “substituted aryl” includes one or two substituents, as defined below.

Some examples of substituents of the above-described aliphatic (and other) moieties of compounds of the invention include, but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, alycyclic, heteroaliphatic, heterocyclic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

The term “neural disorder or condition” includes any and all disorders and conditions that affect the eye and the central nervous system that involve neural degeneration and/or neural cell loss including but not limited to neural injuries associated with hippocampal or RGC degeneration, acute retinal, brain injury, such as angle closure glaucoma, retinal vein occlusions, macular edema, ischemic and hemorrhagic stroke, and traumatic brain injury as well as chronic neurodegenerative retinal or brain disorders such as glaucoma including all forms of primary open angle glaucoma, normal tension glaucoma, as well as retinal ischemias, diabetic retinopathy and diabetic macular edema, age related macular degeneration, retinitis pigmentosa, and Alzheimer's disease (retinal pathology), multiple sclerosis, as well as neurodegenerative brain diseases, such as Alzheimer's disease, Parkinson's disease and ALS.

The term “neuroprotection” as used herein means making a neuron more resistant to a stressor or injury and includes for example inhibiting degeneration, including neurite degeneration and/or further degeneration, promoting survival and/or inhibiting neural cell loss compared to the stressor or injury in the absence of the factor. For example, neurons that are provided one or more of the deuterated analogues of LXB₄ of the present application are more resistant to stress compared to similarly treated neurons not administered the one or more LXB₄ analogues. Neuroprotection may be desired when a subject is at risk of a neurodegenerative disease and under neural stress and includes prophylactic use for example use with subjects with ocular hypertension (risk for glaucoma), diabetes (risk for diabetic retinopathy, macular edema), or subjects with drusen or age-related macular degeneration (exudative or non-exudative forms), as well as for example subjects with a family history of dementia, Alzheimer's disease, Parkinson's disease, etc. Neuroprotection may be desired also after an injury or disease that affects neurons, to protect for example neighbouring neurons from degeneration including neurite degeneration.

The term “central nervous system neurodegeneration and/or neural cell loss” as used herein includes for example degeneration and/or loss of any neurons of the central nervous system including hippocampal neurons, optic neurons and/or retinal ganglion cell (RGC) neurons. Further, the phrase “inhibiting central nervous system neurodegeneration and/or neural cell loss” in the context of administering one or more compounds described herein, means decreasing the number of neurons affected by at least 10%, at least 20%, at least 30%, at least 40% or more compared to the number of neurons affected under similar conditions in the absence of administering the compound.

The term “subject” as used herein includes all members of the animal kingdom including mammals, and suitably refers to humans.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. Treatment methods comprise administering to a subject a therapeutically effective amount of one or more compounds described in the application and optionally consists of a single administration, or alternatively comprises a series of applications. For example, the compounds described herein may be administered at least once a week, about one time per week to about once daily for a given treatment or the compound may be administered one, two, three or four times daily, for example twice daily. The length of the treatment period depends on a variety of factors, such as the severity of the disease, the age of the patient, the concentration, the activity of the compounds described herein, and/or a combination thereof. It will also be appreciated that the effective dosage of the compound used for the treatment or prophylaxis may increase or decrease over the course of a particular treatment or prophylaxis regime. Changes in dosage may result and become apparent by standard diagnostic assays known in the art. In some instances, chronic administration may be required.

As used herein, the phrase, “pharmaceutically acceptable derivative”, denotes any pharmaceutically acceptable salt, ester, salt of such ester, hydrate or solvate of such compound, or any other adduct or derivative which, upon administration to a patient, is capable of providing (directly or indirectly) a compound as otherwise described herein, or a metabolite or residue thereof.

As used herein, the term “protecting group” refers to refers to a moiety that is used for the reversible protection of functional groups during chemical reaction processes to render these functional groups unreactive during chemical reaction processes. It will be appreciated by one of ordinary skill in the art that the synthetic methods, as described herein, utilize a variety of protecting groups. By the term “protecting group”, has used herein, it is meant that a particular functional moiety, e.g., C, O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference. Furthermore, a variety of carbon protecting groups are described in Myers, A.; Kung, D. W.; Zhong, B.; Movassaghi, M.; Kwon, S. J. Am. Chem. Soc. 1999, 121, 8401-8402, the entire contents of which are hereby incorporated by reference. Suitable alcohol protecting groups include, but are not limited to, acetyl, acetonide, allyl, benzoyl, benzyl, b-methoxyethoxymethyl, methoxymethyl, methoxytrityl [(4-methoxyphenyl)diphenylmethyl], p-methoxybenzyl, p-methoxyphenyl, pivaloyl, tetrahydropyranyl, trityl, trimethylsilyl (TMS), triethylsilyl (TES), tert-butyldimethylsilyl (TBDMS), tert-butyldiphenylsilyl, triisopropylsilyl (TIPS), tri-iso-propylsilyloxymethyl, methyl, or ethoxyethyl protecting groups. Suitable alkyne protecting groups include, but are not limited to, TMS, TBDMS, t-hexyldimethylsilyl (TDS), benzyldimethylsilyl (BDMS), biphenyldimethylsilyl, biphenyldiisopropylsilyl (BDIPS) and tris(biphenyl-4-yl)silyl (TBPS).

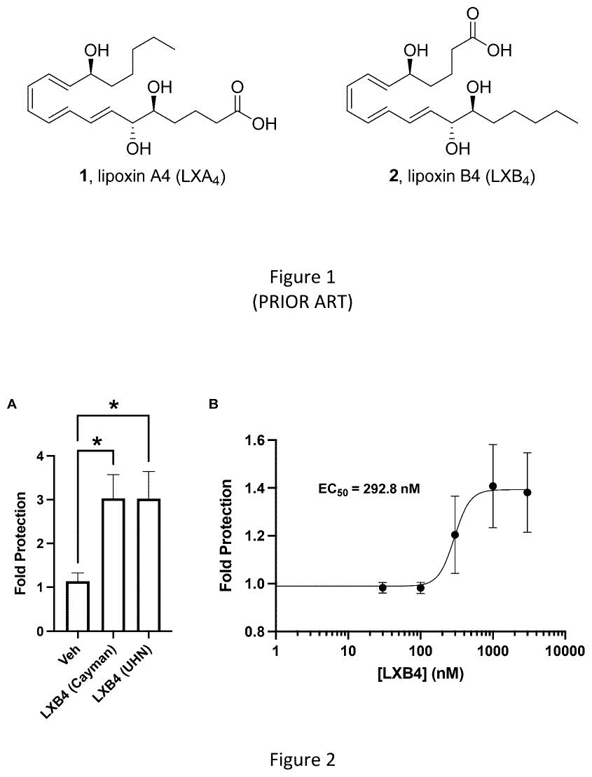

Lipoxins differ in their aliphatic sidechains but share a characteristic and synthetically challenging conjugated tetraene backbone with an E,Z,E,E-configuration (FIG. 1), alongside three key alcohol stereocentres. Formal and total syntheses of LXB₄ have been previously reported using stereospecific,[12c,g] enantioselective[12d,h] and chiral pool synthetic strategies.[12b,e,f,i] However, none provided a robust, scalable and modular synthesis of LXB₄, which is required to both provide unambiguous spectroscopic evidence for its structure, and to enable the future exploration of synthetic analogs for structure-activity relationship (SAR) studies to probe the biological function of LXB₄ in neuroprotection and neurodegeneration.

Based on a retrosynthetic analysis, a Z-selective semi-hydrogenation of alkyne 3 has now been designed by the present inventors to afford LXB₄ (2), as depicted in Scheme 1. Intermediate 3 would be constructed via a Sonogashira coupling of two key fragments, dienyne 4 and vinyl iodide 5. Two routes relying on carbonyl olefinations of functionalized aldehydes were envisioned to dienyne 4, whereas vinyl iodide 5 can be synthesized from commercially available starting materials following a literature route.[15]

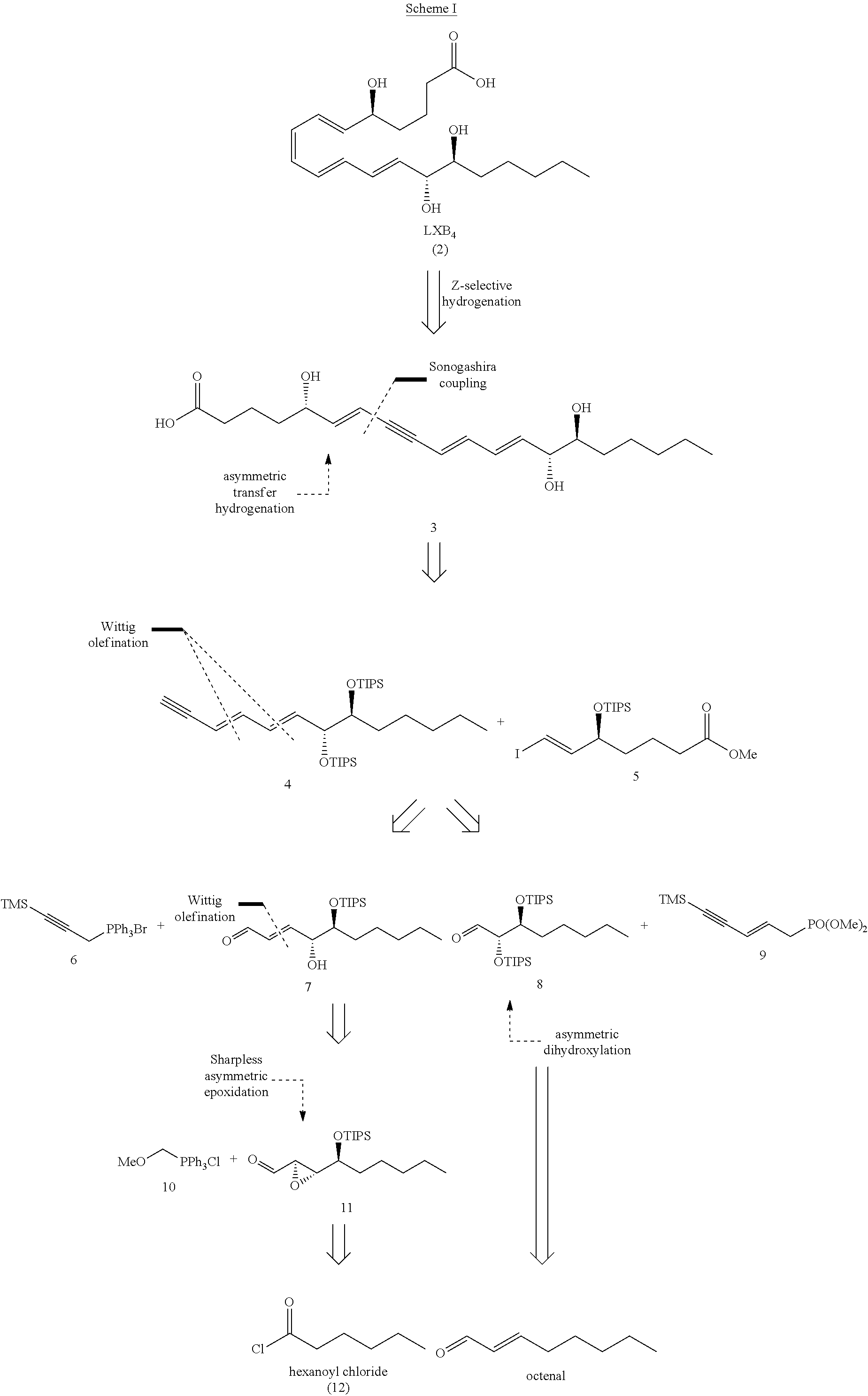

The following discussion relates specifically to use of the synthetic method to produce LXB₄ and deuterated and tritiated analogues thereof. However, it will be appreciated that the general synthetic method can be adapted, as detailed herein, for production of analogues of LXB₄ having the following general structure of Formula I:

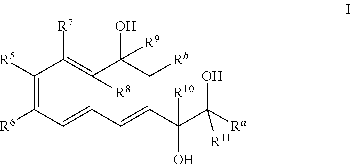

wherein
R⁵—R¹¹ are each independently H, ²H or ³H, optionally where at least one of R⁵—R¹¹ is ²H or ³H,
R^{a} is an optionally substituted alkyl or alkylene, and
R^{b} is an optionally substituted alkyl or alkylene.

In some embodiments, the present process is used to manufacture compounds having one or two deuterium or tritium atoms, with the remainder of the R⁵—R¹¹ groups being hydrogen. In more specific embodiments, the present process is used to manufacture compounds having one or two deuterium atoms, with the remainder of the R⁵—R¹¹ groups being hydrogen. In other embodiments, the present process is used to manufacture LXB₄, which is the compound of Formula I in which each of the R⁵—R¹¹ groups are hydrogen, R^{a} is pentyl and R^{b} is a propionic acid or propionate group, or a salt, ester, hydrate or solvate thereof.

In some examples, the general synthetic method can be adapted, as detailed herein, for production of analogues of LXB₄ having the following general structure of Formula I′:

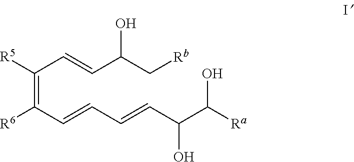

wherein
R⁵ and R⁶ are each independently ²H or ³H,
R^{a} is an optionally substituted alkyl or alkylene, and
R^{b} is an optionally substituted alkyl or alkylene.

As noted above, the present process can be used to produce compounds having one or more deuterium and/or tritium atoms. In these embodiments, as would be well understood by the skilled person, the process is adapted by merely including deuterium and/or tritium in a starting material or introducing the deuterium and/or tritium atom(s) via the reagents used during the process. Alternatively, or in addition, deuterium and/or tritium atom(s) can be added to a product of the reaction using standard techniques.

With reference to Scheme 1, a first route to dienyne 4 depends on a Wittig olefination between phosphonium salt 6 and aldehyde 7, itself the product of a Wittig one-carbon homologation/isomerization cascade on epoxy aldehyde 11. Initially the stereochemistry of the vicinal diol moiety was defined via asymmetric carbonyl reduction and Sharpless asymmetric epoxidation process,[16] and epoxy aldehyde 11 was constructed from hexanoyl chloride 12 using an approach similar to that taken by Kobayashi and co-workers in their syntheses of hydroxyeicosatetraenoic acids (HETEs)[17] and resolvins.[18,19]

An alternative route, also shown in Scheme 1, to dienyne 4 depends on the Horner-Wadsworth-Emmons (HWE) reaction of phosphonate 9 with aldehyde 8; similar olefinations have been used in the syntheses of various other triene and tetraene natural products and derivatives.[20a-g] In turn, the aldehyde 8 could be prepared in a few steps from octenal, with the alcohol stereocentres installed in the first step via an organocatalytic asymmetric dihydroxylation.[21]

With reference to Scheme 2, the synthesis of the advanced intermediate epoxy aldehyde 11 was initially achieved from hexanoyl chloride. Addition of 4-methoxybenzyl (PMB)-protected propargyl alcohol to hexanoyl chloride 12 afforded alkynone 13. In the particular example shown in Scheme 2A, the alkynone 13 was obtained in 76% yield.

The use of readily available acyl halides, such as acyl chlorides, highlights the modularity of this approach, as any aliphatic acyl halide can be incorporated, which allows the synthesis of various analogues of LXB₄. Such analogues can be useful for, for example, in SAR (structure-activity relationship) studies or as therapeutically active compounds. Accordingly, in some embodiments, the formation of an epoxy aldehyde intermediate for use in the present synthetic method for production of an LXB₄ analogue proceeds according to the following reaction:

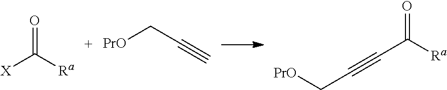

where X is a halide, such as chloride, bromide or iodide; R^{a} is an optionally substituted alkyl or alkylene (e.g., a C₁ to C₁₀ alkyl or alkylene), and Pr is a hydroxy protecting group. In the exemplary synthetic method for producing LXB₄, R^{a} is pentyl.

Asymmetric reduction of 13 using (S)-alpine borane gave the propargyl alcohol 14 in 91% yield and 92% e.e., which was followed by a selective reduction to the E-olefin in 75% yield using Red-Al. The desired epoxy alcohol 16 was then obtained in 77% yield using Sharpless asymmetric epoxidation conditions. The resulting alcohol was protected as the triisopropylsilyl (TIPS) ether 17, which was subjected to sequential PMB removal with DDQ followed by a DMP oxidation to the desired epoxy aldehyde 11 in excellent yields.

A Wittig olefination of aldehyde 11 with (methoxymethyl)triphenylphosphonium chloride 10 furnished the desired hydroxy enal 7 in 50% isolated yield in one step, instead of yielding the expected homologated epoxy aldehyde 19 (Scheme 2A). It was initially proposed to access enal 7 from a base-mediated epoxide ring-opening of 19; however, it was observed that the enol ether product of the olefination reaction 18 was hydrolyzed during aqueous workup or silica gel chromatography to the corresponding enol 20, which presumably undergoes an acid or base catalyzed rearrangement, simultaneously forming the enal system and opening the epoxide to fruitfully give 7 as a single diastereomer in moderate yield. Synthesis of the TMS-protected dienyne fragment 22 was completed by a Wittig olefination on hydroxy enal 7 with commercially available ylide 6, followed by a TIPS protection to furnish the globally protected advanced intermediate (Scheme 2A) (71% over two steps).

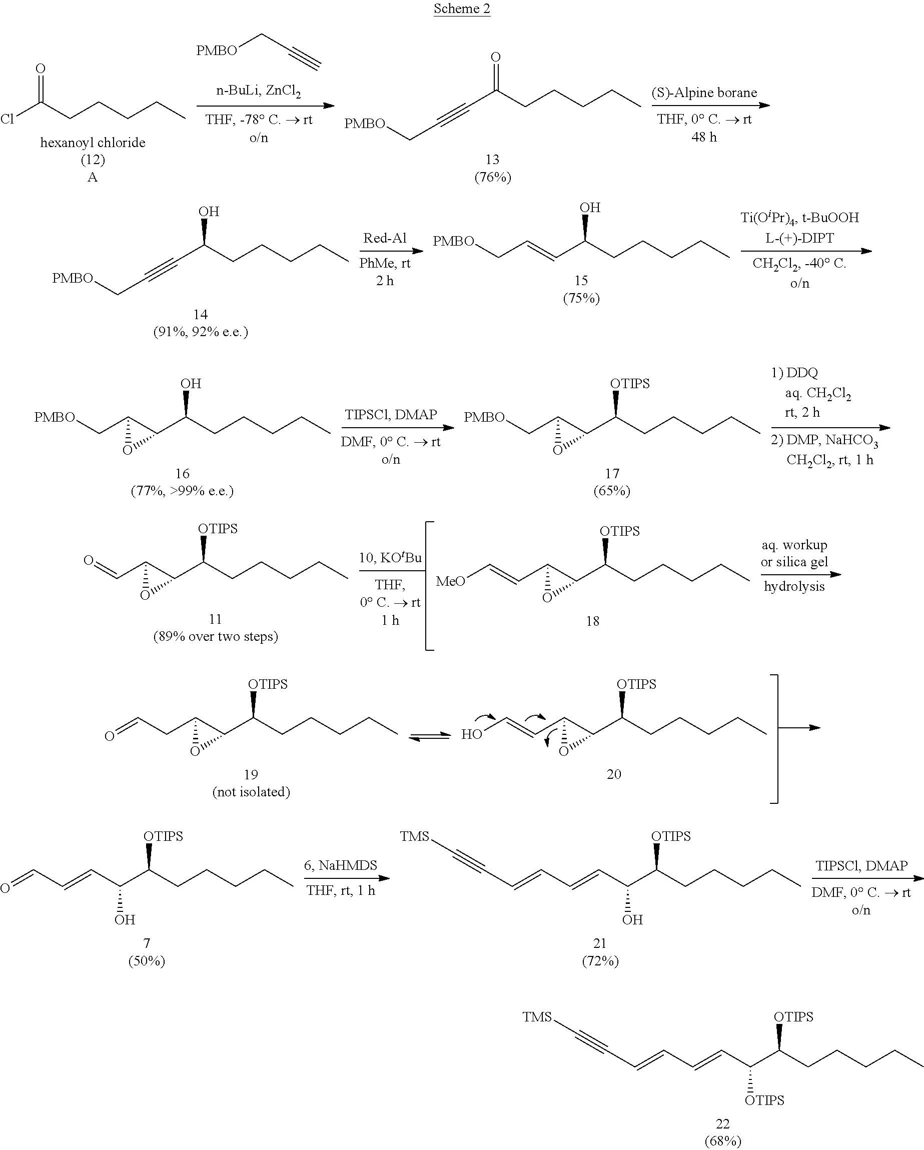

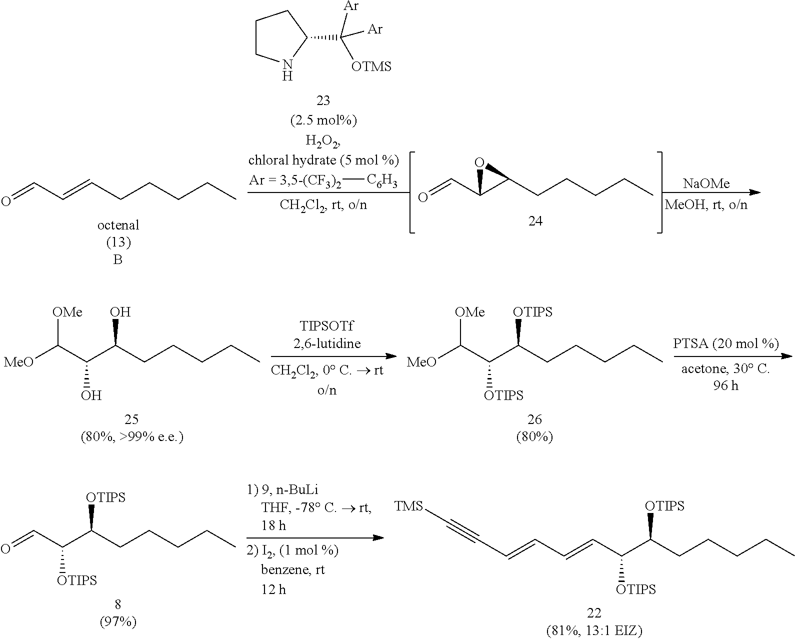

While the synthesis of fragment 22 was achieved from hexanoyl chloride in ten steps, the modest overall yield (6%) prompted the exploration of an alternative route that would reduce the number of steps, while remaining amenable to the incorporation of varying aliphatic chains for synthesis of analogues, for example for future SAR studies. Thus, a route that hinges on an organocatalytic asymmetric dihydroxylation of readily available enals was designed (Scheme 2B).[21]

In some embodiments the starting enal has the structure:

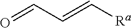

where R^{a} is optionally substituted alkyl or alkylene (e.g., a C₁ to C₁₀ alkyl or alkylene). In the exemplary synthetic method for producing LXB₄, R^{a} is pentyl (i.e., the enal is octenal).

The following discussion describes the reaction starting from octenal as the starting enal. However, is should be appreciated that the process can be performed using other enals as defined above, and that the present synthetic process is not intended to be limited by the use of octenal.

Starting from octenal, the asymmetric dihydroxylation proceeded via an epoxyaldehyde intermediate 24 that was formed upon treatment of 13 with the second generation Jorgensen catalyst (23) and hydrogen peroxide; chloral hydrate acted as a phase transfer catalyst for this first step.[22-24] While epoxide 24 is readily isolable, it can also be directly converted to trans diol 25 by the addition of excess sodium methoxide with concurrent protection of the aldehyde as a dimethyl acetal.[21] Although the conversion of 13 to 25 can be performed as a one-pot procedure (as shown in Scheme 2B), a useful modification was to add a peroxide quenching step to eliminate the possibility of any base-catalyzed epoxidation of trace/unconverted octenal, which would negatively impact the e.e. of 25. Using this modified procedure, 25 was isolated as a single diastereomer in 80% yield and >99% e.e. following a simple aqueous workup. Subsequent protection of the diol with excess TIPSOTf gave the globally protected intermediate 26 in 80% yield. Chemoselective transacetalization of the dimethyl acetal to yield aldehyde 8 was achieved using catalytic PTSA in acetone and very mild heating. The completion of the dienye intermediate from aldehyde 8 proceeded via a HWE reaction with phosphonate 9 followed by an isomerization of the resulting protected dienyne using catalytic iodine.[25] Indeed, similar olefination-isomerization strategies have been successfully used in the synthesis of other conjugated polyenes and lipoxin analogues.[12c,20b,e,f,20h-l] The HWE reaction of aldehyde 8 yielded the desired dienyne in 11:10 E/Z ratio, but isomerization using trace iodine in benzene gave 22 in a 13:1 E/Z ratio and 81% isolated yield over two steps. This alternative route from octenal was achieved in 5 steps with a much improved 50% overall yield.

Having established two viable routes to the desired dienyne intermediate, the carbon backbone of LXB₄ was achieved smoothly with the quantitative removal of the TMS group followed by Sonogashira coupling of enyne 4 with a vinyl halide, such as vinyl iodide 5,[15,20m-q,26] yielding the coupled product 27 in 89% yield (Scheme 3). The TIPS groups were globally removed with TBAF delivering the triol 28 in good yield, followed by hydrolysis of the methyl ester to give the penultimate product 3. The corresponding lactone 29 was also isolated in low quantities but it was easily converted back to the methyl ester 28 or directly hydrolyzed to 3.[12c]

With 3 in-hand, various conditions were explored to the synthesis of LXB₄ via reduction of the internal alkyne (Scheme 5). Unfortunately, various conditions using a Boland reduction of 2 failed to generate the desired E,Z,E,E-conjugated tetraene, contrary to previous reports on similar internal alkynes.[20h,k,27-32] Use of the Boland reduction conditions consistently led to starting material recovery.

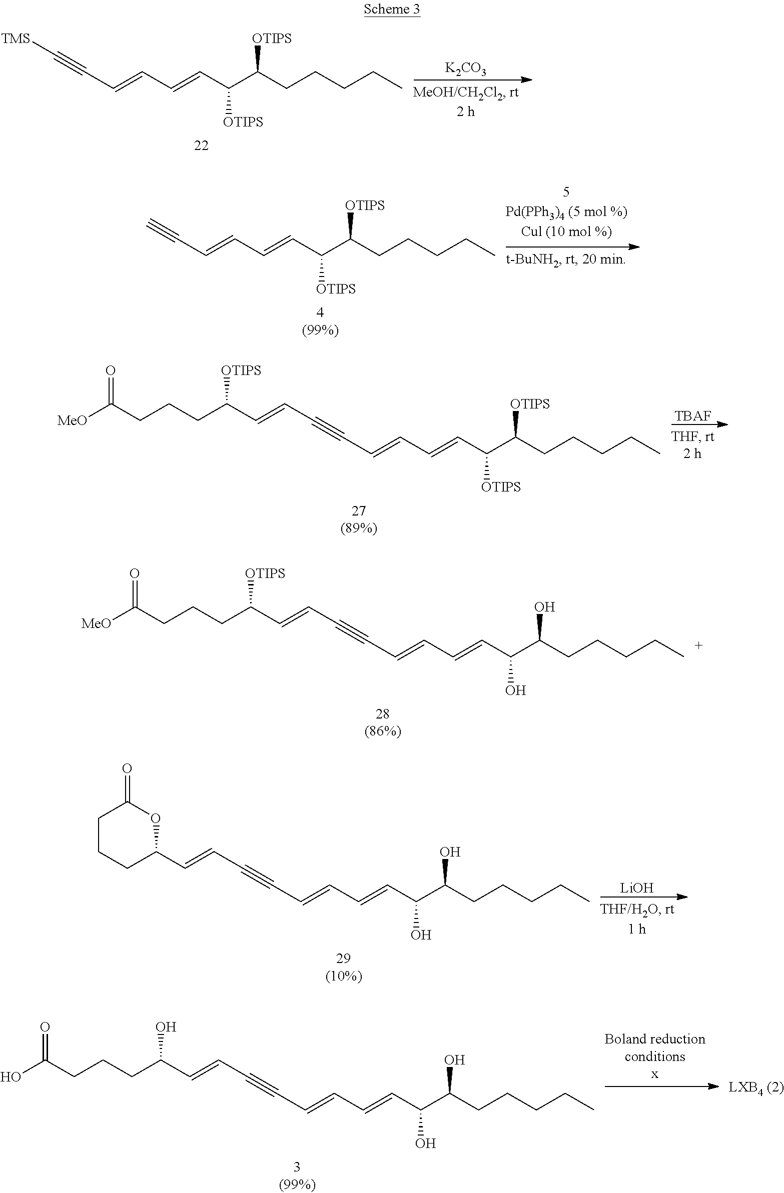

The use of commercially available or readily synthesized vinyl halides in the production of the desired dienyne intermediate, again highlights the modularity of this synthetic approach. Alternative vinyl halides can be used in this step if the process to generate analogues of LXB₄. In some embodiments, the vinyl halide used in the present synthetic process has the following structure:

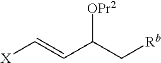

where Pr² is an alcohol protecting group, X is a halide (e.g., Cl, Br or I) and R^{b} is an optionally substituted alkyl or alkylene.

Next, semi-hydrogenation was explored to generate the delicate tetraene system. Unfortunately, no reduction was observed when using P-2 Ni with ethylene diamine.[33,34] As well, the use of various poisoned Pd catalysts, including Lindlar's catalyst, consistently produced a mixture of LXB₄, residual starting material, and over-reduced by-products that were difficult to separate. Attempts to tune the reactivity by modifying the Pd source, additives, and solvent sources were unsuccessful; thus, alternative conditions were explored for installing the tetraene system, inspired by the Karstedt alkyne hydrosilylation/proto-desilylation approach used by Hansen and co-workers in their synthesis of resolvin D1 congeners.[20q,35] Treatment of globally protected 27 with Karstedt's catalyst and ethoxy(dimethyl)silane led to a mixture of hydrosilylated regioisomers 30a and 30b. The regioisomers were subjected to a global silyl deprotection followed by hydrolysis to furnish LXB₄ in 56% isolated yield over three steps via semi-preparative HPLC purification. (Scheme 4).

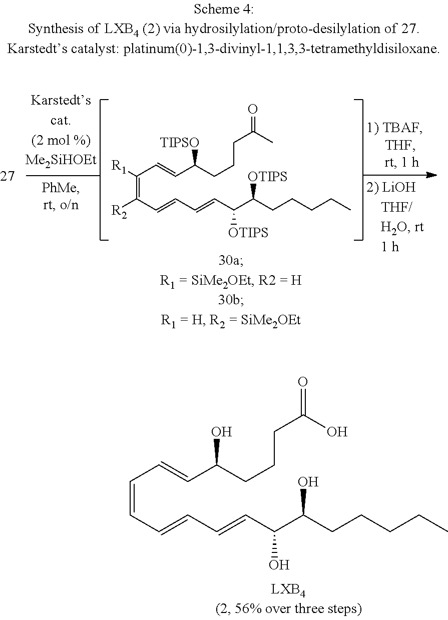

Instability of LXB₄ presented a substantial obstacle its synthesis and purification. LXB₄ is highly unstable to air and light, and rapid degradation was observed when LXB₄ was stored as a solid even at low temperature (−20° C.). Telescoping the final transformations from 27 to 2, preventing light exposure, and storing all crude intermediates in a methanol solution were required to obtain LXB₄ of sufficient purity. LXB₄ could be stored as a solution in methanol or ethanol at −20° C. for several weeks or at −80° C. for one to three months before substantial degradation was observed.

This route is more scalable than any previously published routes. Using this route, it was possible to synthesize 100-200 mg (˜0.25-0.5 mmol) of LXB₄. However, because of the instability of LXB₄, synthesis of 2 on larger scales for longer term storage is not prudent. Accordingly, an alternative embodiment comprises generating the advanced intermediate 27 on an approximately 1 mmol scale. This intermediate can then be stored at −20° C., for at least six months with minimal decomposition, for use in rapid synthesis of pure LXB₄, or a derivative/analogue thereof, for short term use, typically within a few weeks.

Until the present disclosure, incomplete spectroscopic data has been reported for the LXB₄ natural product. ¹H NMR, mass spectrometry, IR, UV, and optical rotation data have been previously reported for the LXB₄ methyl ester (LXB₄-ME), but data provided for the free acid was non-existent. Given the advances in analytical instrumentation since the total synthesis of LXB₄ was first reported, it was possible to obtain extensive spectroscopic data (¹H, ¹³C, COSY, HSQC, NOESY, and HMBC), high-resolution mass spectrometry, HPLC, and optical rotation for this natural product. The spectra for the material obtained using the present synthetic process were consistent with the proposed structure of LXB₄, and also matched the spectral data previously reported for similar tetraene systems, primarily LXB₄-ME,[12c] LXA₄,[3,12g] resolvin D1,[18] and resolvin D2.[29] Furthermore, it was now possible to resolve several NMR peaks at 6.38, 6.28, 5.83, and 5.73 ppm as doublet of doublets which were previously assigned as multiplets in the spectra of LXB₄-ME. To further confirm the structure of LXB4, the material synthesized using the present synthetic process was compared to a commercial sample (Cayman Chemical)[36] and found to be identical by HPLC (mobile phase: acetonitrile+0.1% TFA and MilliQ water+0.1% TFA, gradient from 10:90-95:5, retention time 9.97 min for 2, 9.95 min for commercial sample) and HRMS (ESI HRMS calculated for C20H32O5Na [M+Na]*, predicted m/z 375.2147, found 375.2152, commercial sample 375.2165).[37]

In order to confirm the bioactivity of the synthesized material, its neuroprotective effects were confirmed in a neuronal injury model. Recent clinical failures of a variety of neuroprotection trials have intensified interest in new treatment strategies. Evidence is accumulating that lipoxins and other lipid mediators can have an important impact on neuroinflammation and neuronal survival.[13,38] The present inventors have previously reported that, in addition to their known pro-resolution functions, lipoxins demonstrate an exciting novel protective bioactivity directly on neurons.[14] In particular, LXB₄ was surprisingly 20-fold more potent than LXA₄ in an established neuronal injury model,[14,39] as well as a variety of primary neurons, including cortical and hippocampal cells, and retinal ganglion cells. These broad neuroprotective effects were subsequently correlated with demonstrated LXB₄ efficacy in vivo using both acute and chronic models of retinal ganglion cell death associated with the common neurodegenerative disease glaucoma, a leading cause of vision loss and blindness worldwide.[40-42] Yet the lack of efficient syntheses of LXB₄ coupled with the expensive cost from commercial sources (USD$806/100 μg, Cayman Chemical)[36] have made this new mechanism of action challenging to modify and study. In this regard the presently described synthetic route towards LXB₄, and analogues thereof, provide a benefit for improving production and/or reducing cost of production of LXB₄ and analogues thereof. Therefore, the neuroprotective activity of newly synthesized LXB₄ was compared it against the commercial source to confirm its activity.[36] For this assay, HT22 neuronal cells were pre-treated with LXB₄ from both sources (commercial vs. in-house), followed by a glutamate challenge to induce excitotoxic cell death.[14] Cell viability was then measured using an XTT assay. Both commercial and in-house samples of LXB₄ were significantly neuroprotective in this assay, demonstrating a three-fold recovery in neuronal survival at 1 μM (FIG. 2A). In a parallel dose-response experiment, the EC₅₀ of the newly synthesized LXB₄ was determined to be 292.8 nM (FIG. 2B).

### Radiolabeled Analogues of LXB₄

Radiolabelled LXB₄ can be useful in studies of the target receptor of LXB₄. Such radiolabelled analogues can also be therapeutically useful. In particular, a tritiated analogue of LXB₄ is useful, for example, in vitro target receptor studies, and a deuterated analogue of LXB₄ has therapeutic value as an analogue having the same or similar pharmacological activity as LXB₄ while potentially having improved metabolic stability. Deuterium has recently been used to improve pharmacokinetics of small molecules. Further, such deuterated analogues can be used, for example, for delivery to the eye via a number of different delivery systems.

In one embodiment, there is provided a process for synthesizing radiolabelled, e.g., tritiated LXB₄, by incorporating the radioisotope (deuterium or tritium) in the last synthetic step from advanced intermediate 3 via hydrogenation. This is illustrated using tritium gas (Scheme 5). The transformation resulted in a mixture of unreacted LXB₄, tritiated-LXB₄, and over-reduced LXB₄, allowing isolation of the desired tritiated product 2T via semi-preparative HPLC purification with 96% radiochemical purity. The radiolabelled analogue contained sufficient specific activity of 37 Ci/mmol, exceeding the 20 Ci/mmol required to screen >6,200 human plasma membrane monomers, heterodimers, and secreted proteins within the panel.[43]

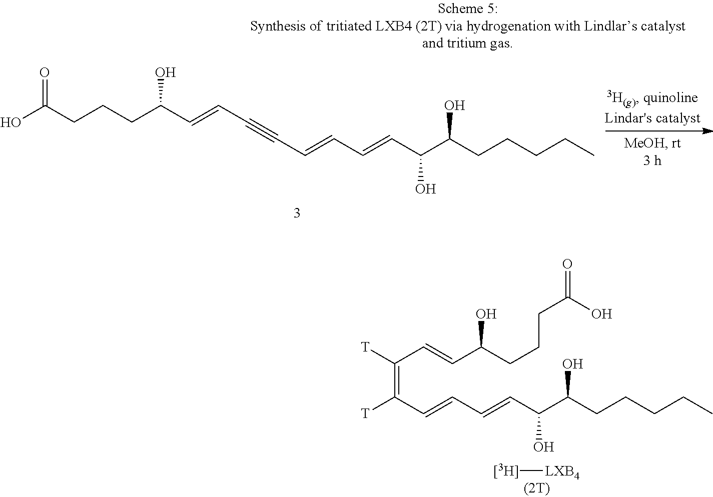

In summary, the present application provides a stereocontrolled and modular total synthesis of LXB₄. The chromatographic data of LXB₄ are consistent with previous reports[1,2,12c,e] and commercial sources.[36] In addition, there is provided unambiguous spectroscopic data to confirm the configurational structure of LXB₄ to be (5S,6E,8Z,10E,12E,14R,15S)-5,14,15-trihydroxyicosa-6,8,10,12-tetraenoic acid.[37]

Two routes to the advanced enyne intermediate 4 were developed, both of which allow for the preparation of future derivatives with varying aliphatic chains. Coupling of 4 with known vinyl iodide 5 successfully allowed the building of the carbon backbone of LXB₄. Moreover, we have explored several strategies for the conversion of the internal alkyne precursors (3 or 27) to the sensitive tetraene system of LXB4. Ultimately, the hydrosilylation/proto-desilylation protocol with Karstedt's catalyst yielded the desired tetraene system of the natural product. Final hydrolysis furnished 100-200 mg of the natural product LXB₄ in 7 steps with an overall yield of 25%, constituting the shortest, most efficient, and scalable route of this natural product to date. With this new route in hand, LXB₄ analogue synthesis, as well as the synthesis of LXB₄-derived chemical biology probes is possible, further investigate the biological function of LXB4 in neuroprotection and neurodegeneration. These analogs can also be used for target receptor and pathway identification purposes.

The present application further provides deuterated and tritiated analogues of LXB₄ having the structure of Formula Ia

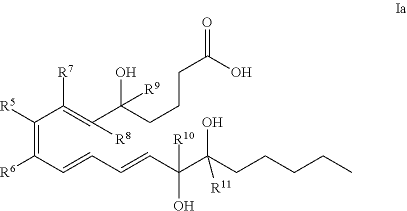

wherein R⁵—R¹¹ are each independently H, ²H or ³H, where at least one of R⁵—R¹¹ is ²H or ³H, or is a pharmaceutically acceptable salt, ester, hydrate, or solvate thereof. Optionally, the compound having the structure of Formula Ia, or the salt, ester, hydrate, or solvate thereof, has the following stereochemistry

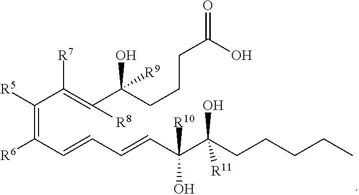

In some embodiments, the present application provides deuterated and tritiated analogues of LXB₄ having the structure of Formula Ia′

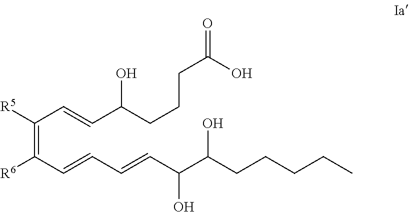

wherein R⁵ and R⁶ are the same and are deuterium or tritium, or is a pharmaceutically acceptable salt, ester, hydrate, or solvate thereof. Optionally, the compound having the structure of Formula Ia′, or the salt, ester, hydrate, or solvate thereof, has the following stereochemistry

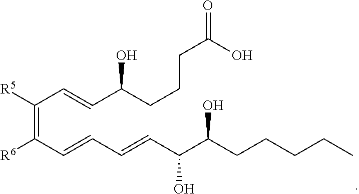

In other embodiments, the present application provides deuterated and tritiated analogues of LXB₄ having the structure of Formula Ia″

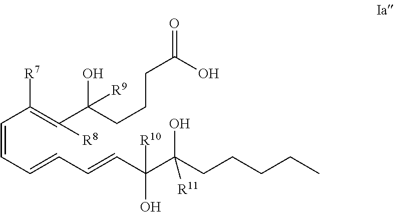

wherein one or two of R⁷—R¹¹ is ²H or ³H, or is a pharmaceutically acceptable salt, ester, hydrate, or solvate thereof, optionally wherein the compound has a structure that is:

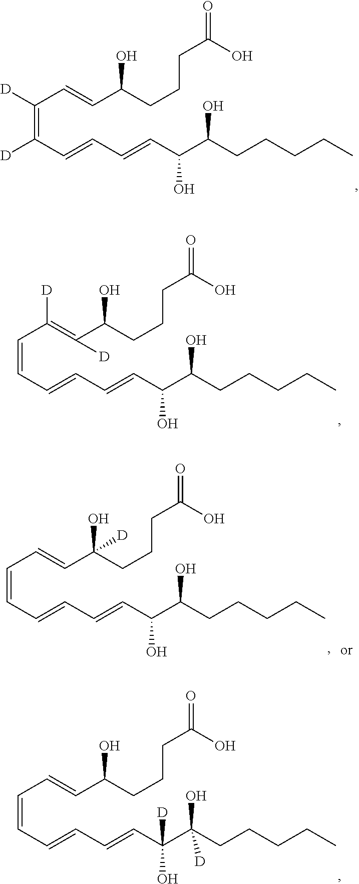

or is a pharmaceutically acceptable salt, ester, hydrate, or solvate thereof.

### Compositions and Methods of Use

Also provided are compositions comprising the compound of Formula Ia or Ia′ in a suitable diluent or excipient.

In some embodiments, there is provided a pharmaceutical composition comprising a compound of Formula Ia in which one or two of R⁷—R¹¹ is deuterium, or a pharmaceutically acceptable salt, ester, hydrate, or solvate thereof, in combination with a pharmaceutically acceptable diluent, excipient, carrier, or combination thereof. In some examples of this embodiment, there is provided a pharmaceutical composition comprising a compound having the structure of Formula Ia′, wherein both R⁵ and R⁶ are deuterium, or a pharmaceutically acceptable salt, ester, hydrate, or solvate thereof, in combination with a pharmaceutically acceptable diluent, excipient, carrier, or combination thereof.

The compound having the structure of Formula Ia or Ia′, or the pharmaceutically acceptable salt, ester, hydrate, or solvate thereof, that does not include tritium and includes one or two deuterium atoms, is useful in the treatment or prevention of diseases and/or conditions associated with neuroinflammation or neurodegeneration, for example, as a neuroprotective agent. Accordingly, there is also provided a method treating a subject susceptible to or having neuroinflammation or neurodegeneration by administering to the subject a compound having the structure of Formula Ia, wherein at least one of R⁵—R¹¹ is deuterium, or of Formula Ia′, wherein both R⁵ and R⁶ are deuterium, or a pharmaceutically acceptable salt, ester, hydrate, or solvate thereof.

It has been found by the present inventors that replacement of deuterium in LXB₄ slows down Phase I metabolism processes of the present compounds thereby improving its overall pharmacokinetics by decreasing clearance of the molecule in vivo. Without wishing to be bound by theory, this may be due the deuterium for hydrogen substitution impeding oxidative metabolism through the primary kinetic isotope effect (KIE). The deuterated compounds of the present application demonstrate very similar pharmacological activity as the parent non-deuterated LXB₄ but, because of their improved pharmacokinetics, they can provide higher and/or prolonged exposure to the subject following administration, in comparison to exposure following LXB₄ administration. The deuterated LXB₄ analogues of the present application are safe and suitable for administration. Further, the improved pharmacokinetics provided by the replacement of one or more hydrogens with deuterium improves their drugability in comparison to the parent LXB₄.

An aspect of this application provides a method or use of providing neuroprotection comprising administering to a subject in need thereof an effective amount of one or more deuterated LXB₄ analogues of Formula Ia or Ia′.

In an embodiment, the neuroprotection is for central nervous system neuroprotection, optionally hippocampal neuroprotection or cortical neuron protection. In another embodiment, the neuroprotection is retinal neuroprotection, optic nerve neuroprotection or RGC neuroprotection.

The neuroprotection can be provided to inhibit neurodegeneration including neurite degeneration and/or to prevent neural cell loss. A subject at risk of developing a disease or condition affecting the central nervous system, retinal neurons, optic nerve or RGC may be a suitable candidate for receiving an effective amount of one or more deuterated LXB₄ analogues of Formula Ia or Ia′.

In an embodiment, the subject has sustained an ischemic and hemorrhagic stroke, or a brain injury such as a traumatic brain injury.

In an embodiment, the one or more deuterated LXB₄ analogue is administered to a subject with ocular hypertension (risk for glaucoma), diabetes (risk for diabetic retinopathy, macular edema), or subjects with drusen or age related macular degeneration (exudative or non-exudative forms). In another embodiment, the one or more deuterated LXB₄ analogue is administered to a subject with a family history of dementia, Alzheimer's disease, Parkinson's disease, etc.

In a further embodiment, the method or use is for inhibiting or preventing RGC degeneration and/or cell loss, optionally resulting from acute injury, the method comprising administering to a subject in need thereof an effective amount of one or more deuterated LXB₄ analogues of Formula Ia or Ia′ such that degeneration and/or cell loss of RGCs is inhibited or prevented.

In an embodiment, the method or use is for treating vision loss, occurring for example related to an acute injury and/or chronic condition.

In an embodiment, the subject is afflicted with an acute retinal injury, such as angle closure glaucoma, retinal vein occlusions, or macular edema.

In an embodiment, the subject is afflicted with a chronic retinal disorder such as glaucoma.

Another aspect includes a method or use of treating a neural disorder or condition, the method comprising administering to a subject in need thereof an effective amount of one or more deuterated LXB₄ analogues of Formula Ia or Ia′.

In an embodiment, the neural disorder or condition is a brain disorder, a retinal neural disorder or retinal neural injury associated with RGC degeneration and/or cell loss. In an embodiment, the neural disorder or condition is a chronic neurodegenerative retinal disorder. [0064] In an embodiment, the chronic neurodegenerative retinal or brain disorder comprises, all forms of primary open angle glaucoma, normal tension glaucoma, retinal ischemias, diabetic retinopathy and macular edema, age related macular degeneration, retinitis pigmentosa, multiple sclerosis, and Alzheimer's disease (retinal pathology), as well as neurodegenerative brain diseases, such as Alzheimer's disease, Parkinson's disease and Amyotrophic Lateral Sclerosis (ALS).

To gain a better understanding of the invention described herein, the following examples are set forth. It should be understood that these examples are for illustrative purposes only. Therefore, they should not limit the scope of this invention in any way.

### EXAMPLES

### Example 1: Synthesis of LXB₄ and Deuterated and Tritiated Analogues of LXB₄, and Testing Thereof

### Methods

Unless otherwise noted, all solvents and reagents were purchased from commercial sources and used as received, or if stated, sparged with a rapid flow of Ar(g) for at least 30 minutes prior to use.

### Chiral HPLC

Chiral HPLC was performed on a Dionex UltiMate 3000™ HPLC system. Chiral compounds were analyzed on a ChiralPak™ IB-3, 150×4.6 mm, 3 μm, chiral HPLC column at room temperature with a flow rate of 1 mL/min. The gradient consisted of eluents A (HPLC-grade hexanes) and B (HPLC-grade isopropanol). An isocratic gradient method of 5% B for 15 minutes was used for chiral analysis.

### Chromatography

Normal-phase flash column chromatography was carried out using Supelco™ technical grade 70-230 mesh silica gel (pore size 60 Å, 63-200 m). Semi-preparative reverse-phase flash column chromatography was carried out on a Gilson Preparative HPLC system. The crude material was injected on an XBridge™ BEH OBD prep column (130 Å, 5 am, 30×150 mm) at room temperature with a flow rate of 15 mL/min. The gradient consisted of eluents A (0.05% NH₄OH in MilliQ™ water) and B (0.05% NH₄OH in HPLC-grade acetonitrile). The gradient method started at 3% B (2 min) followed by a linear gradient from 3 to 33% B over 16 minutes. The mobile phase was then held at 33% B for 5 minutes. The desired product eluted at 19.6 min. For column washing, the strength of the mobile phase was increased in linear gradient from 33% to 95% B over 5 minutes and held at 95% for 2 minutes. The mobile phase was adjusted to 3% in a linear gradient over 5 min, and the column was then equilibrated at 3% B for 2 minutes. Thin-layer chromatography (TLC) was performed on MilliporeSigma silica gel 60 F₂₅₄ coated aluminum-backed TLC sheets and visualized using a UV lamp (254 nm) or KMnO₄ stain.

### Nuclear Magnetic Resonance (NMR) Spectroscopy

Routine ¹H NMR, ¹³C NMR, and 2D spectra were recorded on a Bruker 300 or 400 MHz spectrometer. Spectra for LXB₄ (2) were recorded on a Bruker 700 MHz spectrometer equipped with a 5 mm TCl CryoProbe™. NMR spectra chemical shifts (5) are reported in parts per million (ppm) referenced to residual protonated solvent peak (DMSO-d₆ δ=2.50 ppm, chloroform-d δ=7.26 ppm, methanol-d₄ δ=3.31 ppm). Spectral data is reported as follows: chemical shift, multiplicity (s=singlet, d=doublet, t=triplet, q=quartet, dd=doublet of doublets, dt=doublet of triplets, ddt=doublet of doublet of triplets, dtd=doublet of triplet of doublets, m=multiplet, br=broad), coupling constant (J) in Hertz (Hz), and integration. ¹³C NMR spectra chemical shifts (δ) are reported in parts per million (ppm) and were referenced to carbon resonances in the corresponding NMR solvent (DMSO-d₆ δ=39.5 ppm, chloroform-d δ=77.1 ppm (centre line), methanol-d₄ δ=49.0 ppm).

### Mass Spectrometry

High resolution mass spectra were obtained on a VG 70-250S (double focusing) mass spectrometer at 70 eV, an ABI/Sciex Qstar™ mass spectrometer with ESI source, MS/MS and accurate mass capabilities, or a Waters Xevo™ G2-XS QToF high resolution mass spectrometer.

### Optical Rotation

Optical rotation was measured using a Perkin Elmer Polarimeter™ Model 241 at the sodium D-line (589 nM).

### Syntheses

Procedure from hexanoyl chloride (12)

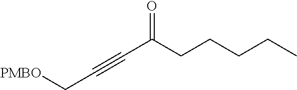

### 1-((4-methoxybenzyl)oxy)non-2-yn-4-one (13)

In a flame-dried 2-neck 250 mL round-bottom flask equipped with a stir bar was added PMB-propargyl alcohol (3.0 g, 17.0 mmol, 1.0 equiv.) dissolved in anhydrous THF (85 mL, 0.2M) at room temperature under Ar atmosphere. The solution was cooled to −78° C. with a dry ice/acetone bath. n-BuLi* (2.5 M in hexanes, 8.2 mL, 20.4 mmol, 1.2 equiv.) was added dropwise at −78° C. over 5 minutes. The resulting yellow mixture was allowed to stir at −78° C. for 30 minutes. ZnCl₂ (0.5M in THF, 40.9 mL, 20.4 mmol, 1.2 equiv.) was then added dropwise at −78° C. over 5 minutes. The resulting clear mixture was allowed to stir at −78° C. for another 30 minutes. Hexanoyl chloride (2.86 mL, 20.4 mmol, 1.2 equiv.) was then added in one portion to the mixture at −78° C. The cooling bath was removed, and the resulting mixture was allowed to slowly warm to room temperature and stir overnight. Upon completion as indicated by TLC analysis, the reaction mixture was quenched with saturated NH₄Cl solution and diluted with ethyl acetate. The two phases were separated, and the aqueous layer was extracted twice with ethyl acetate. The combined organic layers were dried over Na₂SO₄, filtered, and concentrated in vacuo. The crude residue was purified by flash column chromatography on silica gel (hexanes/diethyl ether 9:1) to afford alkynone (3.2 g, 76% yield) as a pale-yellow oil. *n-BuLi should be titrated before use.

¹H NMR (400 MHz, CDCl₃) δ 7.31-7.26 (m, 2H), 6.89 (dt, J=8.6, 2.8 Hz, 2H), 4.55 (s, 2H), 4.29 (s, 2H), 3.81 (s, 3H), 2.57 (t, J=7.5 Hz, 2H), 1.72-1.64 (m, 2H), 1.35-1.29 (m, 4H), 0.90 (d, J=7.0 Hz, 3H).

¹³C NMR: 187.8, 159.7, 130.0, 128.8, 114.0, 87.7, 85.4, 71.8, 56.7, 55.4, 45.5, 31.2, 24.5, 23.7, 22.5, 14.0

DART HRMS calculated for C₁₇H₂₂O₃(M)⁺: 274.1569, found 274.1564.

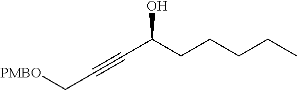

### (S)-1-((4-methoxybenzyl)oxy)non-2-yn-4-ol (14)

In a flame-dried 100 mL round-bottom flask equipped with a stir bar was added alkynone (1.5 g, 5.47 mmol, 1.0 equiv.) dissolved in anhydrous THF (11 mL, 0.5M). The mixture was cooled to 0° C. and (S)-Alpine borane (2.98 mL, 10.9 mmol, 2.0 equiv.) was added dropwise over 10 minutes at 0° C. The cooling bath was removed and the reaction mixture was allowed to slowly warm to room temperature and stir at room temperature for 48 h. Upon completion as indicated by TLC analysis, the solvent was removed in vacuo. The resulting residue was diluted with 60 mL of diethyl ether and cooled to 0° C. Ethanolamine (1.1 mL) was added dropwise at 0° C. and the resulting cloudy mixture was allowed to stir at 0° C. for 1 h. The emulsion was filtered over a pad of Celite and concentrated in vacuo. The crude residue was purified by flash column chromatography on silica gel (hexanes/ethyl acetate 9:1) to afford propargyl alcohol (1.37 g, 91% yield) as a pale-yellow oil; 91.6% e.e. determined by HPLC [ChiralPak IB-3; hexane/i-PrOH=95:5, 1 mL min⁻¹, 23° C.; t_{R} (min)=6.47 (minor), 8.02 (major).

¹H NMR (400 MHz, CDCl₃) δ 7.28 (dd, J=8.7, 3.1 Hz, 2H), 6.88 (dt, J=8.7, 2.9 Hz, 2H), 4.52 (s, 2H), 4.46-4.38 (m, 1H), 4.17 (d, J=1.7 Hz, 2H), 3.81 (s, 3H), 1.86-1.83 (m, 1H), 1.75-1.67 (m, 2H), 1.49-1.42 (m, 2H), 1.36-1.29 (m, 4H), 0.91 (d, J=6.8 Hz, 3H).

¹³C NMR: 159.5, 129.9, 129.5, 113.9, 87.7, 80.9, 71.3, 62.7, 57.1, 55.4, 37.8, 31.6, 25.0, 22.7, 14.1

DART HRMS calculated for C₁₇H₂₈O₃N (M+NH₄)⁺: 294.2069, found 294.2073.

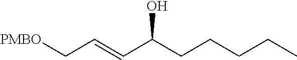

### (S,E)-1-((4-methoxybenzyl)oxy)non-2-en-4-ol (15)

In a 250 mL round-bottom flask equipped with a stir bar was added secondary propargyl alcohol (1.1 g, 3.98 mmol, 1.0 equiv.) dissolved in toluene (8.0 mL, 0.5M) under Ar atmosphere. The solution was cooled to 0° C. with an ice/water bath where Red-Al (3.6 M in PhMe, 2.76 mL, 9.95 mmol, 2.5 equiv.) was added. The cooling bath was then removed, and the reaction mixture was allowed to slowly warm to room temperature and stirred for 2 hours. Upon completion as indicated by TLC analysis, the reaction was quenched with the addition of H₂O (1.1 mL), NaF (2.51 g, 59.7 mmol, 15 equiv.), and Celite (3.0 g). The suspension was stirred at room temperature for 30 minutes and then filtered through a pad of Celite. The resulting solution was concentrated in vacuo to afford the crude mixture. The crude residue was purified by flash column chromatography on silica gel (hexanes/ethyl acetate 7:3) to afford secondary allylic alcohol (830 mg, 75% yield) as a pale-yellow oil.

¹H NMR (400 MHz, MeOD) δ 7.27 (dt, J=8.7, 2.9 Hz, 2H), 6.88 (dt, J=8.8, 2.7 Hz, 2H), 5.84-5.70 (m, 2H), 4.45 (s, 2H), 4.17-4.07 (m, 1H), 4.03-3.96 (m, 2H), 3.80 (s, 3H), 1.59-1.25 (m, 9H), 0.88 (t, J=7.1 Hz, 3H).

¹³C NMR: 159.3, 136.3, 130.4, 129.5, 127.2, 113.9, 72.5, 72.0, 70.0, 55.4, 37.2, 31.9, 25.2, 22.7, 14.2

DART HRMS calculated for C₁₇H₂₉O₃N (M+NH₄)⁺: 296.2226, found 296.2216.

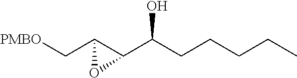

### (S)-1-((2S,3S)-3-(((4-methoxybenzyl)oxy)methyl)oxiran-2-yl)hexan-1-ol (16)

In a 100 mL round-bottom flask equipped with a stir bar was added 4A MS (½ by weight) in anhydrous dichloromethane (6 mL) under Ar atmosphere. The mixture was cooled to −40° C. using an acetonitrile/dry ice bath where Ti(OiPr)₄ (0.85 mL, 2.87 mmol, 1.0 equiv.) and L-(+)-DIPT (0.71 mL, 3.45 mmol, 1.2 equiv.) were added in one portion. The resulting mixture was stirred at −40° C. for 30 minutes. A solution of secondary allylic alcohol (800 mg, 2.87 mmol, 1.0 equiv.) in dichloromethane (12 mL) was slowly added at −40° C. and stirred at −40° C. for another 30 minutes. TBHP (5.5 M in dec, 0.78 mL, 4.31 mmol, 1.5 equiv.) was added dropwise to the reaction mixture at −40° C. and stirred at −40° C. for 2 hours. The reaction flask was then placed in the freezer overnight. Upon completion as indicated by TLC analysis, the reaction mixture was quenched by pouring over a cool solution of acetone/water (10:2 mL) at 0° C. and washed with dichloromethane. The resulting mixture was stirred at room temperature for 1 hour. The yellow suspension was then filtered through a pad of Celite and the filtrate was concentrated in vacuo. The crude residue was purified by flash column chromatography on silica gel (hexanes/ethyl acetate 9:1) to afford the epoxy alcohol product (650 mg, 77% yield) as a yellow oil; >99% e.e. determined by HPLC [ChiralPak IB-3; hexane/i-PrOH=95:5, 1 mL min-1, 23° C.; t_{R} (min)=9.23 (major), 9.89 (minor).

¹H NMR (400 MHz, CDCl₃) δ 7.33-7.25 (m, 2H), 6.95-6.86 (m, 2H), 4.55 (d, J=11.5 Hz, 2H), 4.51 (d, J=11.3 Hz, 2H), 3.86 (dq, J=7.0, 3.0 Hz, 1H), 3.83 (s, 3H), 3.78 (dd, J=11.6, 2.8 Hz, 1H), 3.48 (dd, J=11.6, 5.7 Hz, 1H), 3.28 (dt, J=5.4, 2.6 Hz, 1H), 2.97 (dd, J=3.2, 2.4 Hz, 1H), 1.88 (d, J=2.5 Hz, 1H), 1.60-1.27 (m, 9H), 0.95-0.87 (m, 3H).

¹³C NMR (contains DIPT, peaks matching lit. DIPT spectrum excluded): 159.4, 130.0, 129.5, 113.9, 73.1, 69.5, 68.3, 58.2, 55.4, 53.6, 33.5, 31.9, 25.0, 22.7, 14.2

DART HRMS calculated for C₁₇H₃₀O₄N (M+NH₄)⁺: 312.2175, found 312.2169

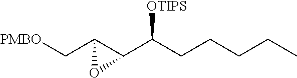

### Triisopropyl(((S)-1-((2R,3S)-3-(((4-methoxybenzyl)oxy)methyl)oxiran-2-yl)hexyl)oxy)silane (17)

In a 250 mL round-bottom flask equipped with a stir bar was added epoxy alcohol (1.75 g, 5.94 mmol, 1.0 equiv.) dissolved in dimethylformamide (6 mL, 1 M) at room temperature. The solution was cooled to 0° C. in an ice/water bath where DMAP (2.2 g, 17.8 mmol, 2.5 equiv.) and TIPSCl (1.91 mL, 8.9 mmol, 1.5 equiv.) were added sequentially. The mixture was allowed to stir at 0° C. for 30 minutes and then at room temperature overnight. Upon completion as indicated by TLC analysis, the reaction was quenched by the addition of saturated NaHCO₃ and diluted with ethyl acetate. The two layers were separated, and the aqueous layer was extracted twice with ethyl acetate. The combined organic layers were dried over Na₂SO₄, filtered, and concentrated in vacuo. The crude residue was purified by flash column chromatography on silica gel (hexanes/ethyl acetate 19:1) to afford the bis-protected epoxy alcohol (1.73 g, 65% yield) as a yellow oil.

¹H NMR (400 MHz, CDCl₃) δ 7.27 (d, J=6.9 Hz, 4H), 6.87 (dt, J=8.5, 3.0 Hz, 2H), 4.53 (d, J=11.5 Hz, 1H), 4.48 (d, J=11.5 Hz, 1H), 3.83-3.77 (m, 4H), 3.73 (q, J=5.2 Hz, 1H), 3.38 (dd, J=11.7, 6.0 Hz, 1H), 3.16 (dt, J=6.1, 2.3 Hz, 1H), 2.82 (dd, J=5.2, 2.2 Hz, 1H), 1.61 (dt, J=10.0, 5.1 Hz, 2H), 1.53-1.26 (m, 6H), 1.04 (m, 21H), 0.88 (t, J=6.9 Hz, 3H).

¹³C NMR: 159.3, 130.2, 129.5, 113.9, 73.0, 71.2, 70.0, 55.7, 55.4, 36.0, 32.3, 23.8, 22.7, 18.2, 18.2, 14.2, 12.6

DART HRMS calculated for C₂₆H₅₀O₄NSi (M+NH₄)⁺: 468.3509, found 468.3502

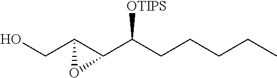

### ((2S,3R)-3-((S)-1-((triisopropylsilyl)oxy)hexyl)oxiran-2-yl)methanol

In a 250 mL round-bottom flask equipped with a stir bar was added PMB epoxy ether (1.55 g, 3.44 mmol, 1.0 equiv.) dissolved in dichloromethane (35 mL, 0.1 M) and a pH 6.8 phosphate buffer solution (35 mL) at room temperature. DDQ (1.17 g, 5.16 mmol, 1.5 equiv.) was subsequently added to the mixture and the resulting solution was allowed to stir at room temperature for 2 hours. Upon completion as indicated by TLC analysis, the reaction was quenched with adding saturated NaHCO₃ and diluted with dichloromethane. The two layers were then separated, and the aqueous layer was extracted twice with dichloromethane. The combined organic layers were dried over Na₂SO₄, filtered, and concentrated in vacuo. The crude residue was purified by flash column chromatography on silica gel (hexanes/ethyl acetate 9:1) to afford the primary epoxy alcohol (1 g, 89% yield) as a pale-yellow oil.

¹H NMR (400 MHz, CDCl₃) δ 3.97 (ddd, J=12.7, 5.4, 2.2 Hz, 1H), 3.81 (q, J=5.0 Hz, 1H), 3.62 (ddd, J=12.3, 7.5, 4.3 Hz, 1H), 3.16 (dt, J=4.4, 2.3 Hz, 1H), 2.96 (dd, J=4.9, 2.3 Hz, 1H), 1.70-1.59 (m, 2H), 1.53-1.25 (m, 6H), 1.05 (s, 21H), 0.93-0.85 (m, 3H).

¹³C NMR: 70.8, 61.5, 58.0, 56.5, 35.9, 32.3, 23.8, 22.7, 18.2, 18.1, 14.2, 12.6

DART HRMS calculated for C₁₈H₃₉O₃Si (M+H)⁺: 331.2668, found 331.2663

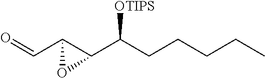

### (2R,3R)-3-((S)-1-((triisopropylsilyl)oxy)hexyl)oxiran-2-carbaldehyde (11)

In a 100 mL round-bottom flask equipped with a stir bar was added primary epoxy alcohol (800 mg, 2.42 mmol, 1.0 equiv.) dissolved in dichloromethane (25 mL, 0.1 M) at room temperature under Ar atmosphere. NaHCO₃ (244 mg, 2.90 mmol, 1.2 equiv.) and DMP (1.23 g, 2.90 mmol, 1.2 equiv.) were added sequentially to the solution at room temperature. The resulting mixture was allowed to stir at room temperature for 1 hour. Upon completion as indicated by TLC analysis, the solvent was removed in vacuo and was directly loaded onto the column for purification. The crude residue was purified by flash column chromatography on silica gel (hexanes/ethyl acetate 9:1) to afford epoxy aldehyde (788 mg, 99% yield) as a pale-yellow oil.

¹H NMR (400 MHz, CDCl₃) δ 9.08 (d, J=6.3 Hz, 1H), 4.00 (q, J=4.8 Hz, 1H), 3.41 (dd, J=6.4, 2.0 Hz, 1H), 3.23 (dd, J=3.8, 1.9 Hz, 1H), 1.69-1.60 (m, 2H), 1.51-1.25 (m, 6H), 1.05 (m, 21H), 0.89 (t, J=7.0 Hz, 3H).

¹³C NMR: 198.6, 69.5, 59.2, 56.7, 35.8, 32.2, 23.7, 22.7, 18.2, 18.2, 14.1, 12.6

DART HRMS calculated for C₁₈H₃₇O₃Si (M+H)⁺: 329.2512, found 331.2518

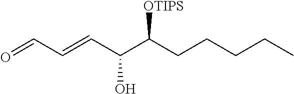

### (4R,5S,E)-4-hydroxyl-5-((triisopropylsilyl)oxy)dec-2-enal (7)

In a 250 mL round-bottom flask equipped with a stir bar was added (methoxymethyl)triphenylphosphonium chloride (9.83 g, 28.7 mmol, 2.5 equiv.) in anhydrous THF (57 mL) at room temperature under Ar atmosphere. Cool the white suspension to 0° C. with an ice/water bath followed by the dropwise addition of a solution of KO^{t}Bu (3.22 g, 28.7 mmol, 2.5 equiv.) in anhydrous THF (29 mL) at 0° C. (solution turned bright orange/red). The resulting mixture was allowed to stir at 0° C. for 30 minutes, at which point a solution of epoxy aldehyde (3.8 g, 11.5 mmol, 1.0 equiv.) in anhydrous THF (23 mL) was slowly added to the ylide mixture at 0° C. The resulting mixture was allowed to stir at 0° C. for 5 minutes until the ice/water bath was removed to gradually warm the solution to room temperature. The mixture was allowed to stir at room temperature for 1 h. Upon completion by TLC analysis, the solvent was removed in vacuo and was directly loaded onto the column for purification. The crude residue was purified by flash column chromatography on silica gel (hexanes/ethyl acetate 9:1) to afford the unprotected enal alcohol (1.93 g, 50% yield) as a pale-yellow oil, a single diastereomer by ¹H NMR.

¹H NMR (700 MHz, CDCl₃) δ 9.60 (d, J=7.9 Hz, 1H), 6.82 (dd, J=15.7, 4.7 Hz, 1H), 6.38 (ddd, J=15.7, 7.9, 1.7 Hz, 1H), 4.48 (tdd, J=4.9, 3.4, 1.7 Hz, 1H), 4.02 (td, J=6.2, 3.5 Hz, 1H), 2.59 (d, J=4.8 Hz, 1H), 1.49-1.34 (m, 2H), 1.32-1.23 (m, 6H), 1.14-1.06 (m, 21H), 0.87 (t, J=7.1 Hz, 3H).

¹³C NMR (176 MHz, CDCl₃) δ 193.5, 154.5, 132.5, 75.3, 74.2, 32.9, 32.2, 25.2, 22.7, 18.3, 18.3, 14.1.

ESI HRMS calculated for C₁₉H₃₈O₃SiNa (M+Na)⁺: 365.2482, found 365.2479

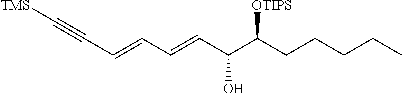

### (3E,5E,7R,8S)-8-((triisopropylsilyl)oxy)-1-(trimethylsilyl)trideca-3,5-dien-1-yn-7-ol (21)

In a 500 mL round-bottom flask equipped with a stir bar was added TMS-propargyltriphenylphosphonium bromide (3.83 g, 8.45 mmol, 1.5 equiv.) in anhydrous THF (120 mL) at room temperature under Ar atmosphere. NaHMDS (1 M in THF, 8.45 mL, 8.45 mmol, 1.5 equiv.) was added dropwise to the suspension at room temperature where the mixture turned yellow. The resulting mixture was allowed to stir at room temperature for 30 minutes. A solution of enal alcohol (1.93 g, 5.63 mmol, 1.0 equiv) in anhydrous THF (70 mL) was slowly added to the mixture at room temperature. The resulting mixture was allowed to stir at room temperature for 1 h. Upon completion by TLC analysis, deionized water was added to the mixture and diluted with diethyl ether. After separation, the aqueous layer was extracted twice with diethyl ether. The combined organic layers were dried over Na₂SO₄, filtered, and concentrated in vacuo. The crude residue was purified by flash column chromatography on silica gel (hexanes/ethyl acetate 95:5) to afford the TMS-protected dienyne (1.72 g, 72% yield) as a yellow oil in a 3:1 E/Z inseparable mixture and was carried on to the next TIPS protection step as a mixture.

¹H NMR (700 MHz, CDCl₃) δ 6.64 (dd, J=15.6, 10.7 Hz, 1H), 6.18 (dd, J=15.4, 10.8 Hz, 1H), 5.82 (dd, J=15.3, 7.8 Hz, 1H), 5.61 (d, J=15.6 Hz, 1H), 4.17 (dd, J=7.8, 3.4 Hz, 1H), 3.67 (dt, J=8.4, 3.9 Hz, 1H), 2.46-2.42 (br m, 1H), 1.51-1.22 (m, 8H), 1.11-1.01 (m, 21H), 0.90-0.86 (m, 3H), 0.20 (d, J=10.4 Hz, 12H).

¹³C NMR (176 MHz, CDCl₃) δ 142.1, 135.0, 131.5, 111.5, 104.3, 97.7, 76.6, 75.3, 32.2, 32.0, 25.7, 22.7, 18.3, 18.1, 14.2, 12.8, 12.4, 0.0.

ESI HRMS calculated for C₂₅H₄₈O₂SiNa (M+Na)⁺: 459.3091, found 359.3072

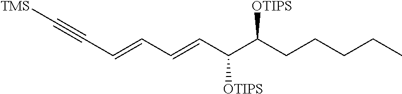

### (5S,6R)-3,3,8,8-tetraisopropyl-2,9-dimethyl-5-pentyl-6-((1E,3E)-6-(trimethylsilyl)hexa-1,3-dien-5-yn-1-yl)-4,7-dioxa-3,8-disiladecane (22)

In a 100 mL round-bottom flask equipped with a stir bar was added dienyne alcohol (1.72 g, 3.94 mmol, 1.0 equiv.) dissolved in dimethylformamide (4 mL, 1 M) at room temperature. The solution was cooled to 0° C. in an ice/water bath where DMAP (1.2 g, 9.84 mmol, 2.5 equiv.) and TIPSCl (1.26 mL, 5.9 mmol, 1.5 equiv.) were added sequentially. The mixture was allowed to stir at 0° C. for 30 minutes and then at room temperature overnight. Upon completion as indicated by TLC analysis, the reaction was quenched by the addition of saturated NaHCO₃ and diluted with ethyl acetate. The two layers were separated, and the aqueous layer was extracted twice with ethyl acetate. The combined organic layers were dried over Na₂SO₄, filtered, and concentrated in vacuo. The crude residue was purified by flash column chromatography on silica gel (hexanes/ethyl acetate 19:1) to afford the bis-protected diol dienyne (1.59 g, 68% yield) as a yellow oil in a 3:1 E/Z inseparable mixture carried forward from the previous step.

¹H NMR (400 MHz, CDCl₃) δ 6.65 (dd, J=15.6, 10.7 Hz, 1H), 6.11 (dd, J=15.4, 10.8 Hz, 1H), 5.88 (dd, J=15.5, 7.8 Hz, 1H), 5.57 (d, J=15.7 Hz, 1H), 4.15 (dd, J=7.8, 3.0 Hz, 1H), 3.87-3.81 (m, 1H), 1.50-1.43 (m, 2H), 1.34-1.23 (m, 6H), 1.06-1.01 (m, 42H), 0.88 (t, J=7.1 Hz, 3H), 0.19 (s, 9H).

¹³C NMR (101 MHz, CDCl₃) δ 142.6, 137.9, 130.4, 110.4, 104.5, 96.9, 77.4, 76.8, 34.6, 32.2, 24.9, 22.6, 18.3, 18.3, 18.2, 18.1, 14.1, 12.8, 12.5, 0.0.

DART HRMS calculated for C₃₄H₆₉O₂Si₃ (M+H)⁺: 593.4600 found 593.4593.

Procedure from Octenal

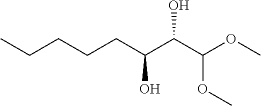

### (2S,3S)-1,1-dimethoxyoctane-2,3-diol (25)

Octenal (0.30 mL, 2.0 mmol, 1.0 equiv.) was dissolved in 4 mL CH₂Cl₂ in a screw-cap vial charged with a stir bar. Then, (R)-2-(bis(3,5-bis(trifluoromethyl)phenyl)((trimethylsilyl)oxy)methyl) pyrrolidine (0.030 g, 0.05 mmol, 0.025 equiv.) and chloral hydrate (0.017 g, 0.10 mmol, 0.05 equiv.) were added and the mixture was allowed to stir for approximately 5 minutes. Next, hydrogen peroxide (30-35% in H₂O, 0.30 mL, approx. 3.0 mmol, 1.5 equiv.) was added. The vial was sealed, and the reaction was vigorously stirred for 20 hours. Then, 1.0 mL of water was added, followed by 1.0 mL saturated sodium sulfite, and the reactions were vigorously stirred for 10 minutes to ensure complete consumption of the remaining peroxide. The organic layer was removed, and the aqueous layer extracted with CH₂Cl₂ (2×2 mL) and the combined organic fractions were combined and concentrated in vacuo to give a fragrant light-yellow oil. To this was added 40 mL of 0.5 M NaOMe in MeOH (10.0 equiv.) and the reaction was stirred another 18 hours. Then, 20 mL of water was added to quench the reaction. The mixture was extracted with CH₂Cl₂ (3×20 mL), the combined organic layers washed with brine, dried over Na₂SO₄, and solvent removed in vacuo to give 25 (0.33 g, 80% yield) as a light-yellow oil that was used without further purification.

¹H NMR (400 MHz, CDCl₃) δ 4.40 (d, J=5.7 Hz, 1H), 3.73-3.67 (m, 1H), 3.54 (td, J=5.6, 3.8 Hz, 1H), 3.47 (s, 3H), 3.46 (s, 3H), 2.55 (d, J=4.3 Hz, 1H), 2.37 (s, 1H), 1.64-1.43 (m, 3H), 1.39-1.24 (m, 5H), 0.89 (t, J=6.8 Hz, 3H). All data are consistent with the literature.^{[1]}

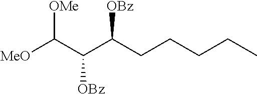

### (2S,3S)-1,1-dimethoxyoctane-2,3-diyl dibenzoate

*The dibenzoate was synthesized and used to determine the % e.e. of the asymmetric dihydroxylation.*

(2S,3S)-1,1-dimethoxyoctane-2,3-diol (0.113 g, 0.55 mmol, 1.0 equiv.) was dissolved in 5.5 mL CH₂Cl₂ in a 50 mL single-neck round-bottom flask. To this was added triethylamine (0.38 mL, 2.75 mmol, 5.0 equiv.) and DMAP (6.7 mg, 0.055 mmol, 0.1 equiv.). The flask was sealed under argon and cooled to 0° C. Then, benzoyl chloride (0.16 mL, 1.38 mmol, 2.5 equiv.) was added in one portion and the reaction was stirred overnight with warming to room temperature. The reaction was quenched with 5 mL saturated NH₄Cl and the organic layer was reserved. The aqueous layer was extracted with Et₂O (3×10 mL). The combined organic layers were washed with brine, dried over MgSO₄, and concentrated in vacuo to give a yellow oil that was further purified using silica gel chromatography (0-20% Et₂O in hexanes). Concentration of the desired fractions yielded 0.123 g (54% yield) of the dibenzoate as a clear, colourless oil that crystallizes upon standing in a freezer; >99% e.e. determined by HPLC [ChiralPak IB-3; hexane/i-PrOH=95:5, 1 mL min⁻¹, 23° C.; t_{R} (min)=2.94 (major), 3.15 (minor).

¹H NMR (400 MHz, CDCl₃) δ 8.05 (dd, J=8.4, 1.3 Hz, 2H), 7.98 (dd, J=8.4, 1.3 Hz, 2H), 7.61-7.56 (m, 1H), 7.56-7.51 (m, 1H), 7.46 (t, J=7.7 Hz, 2H), 7.40 (t, J=7.7 Hz, 2H), 5.62 (dd, J=6.8, 3.4 Hz, 1H), 5.51 (dt, J=9.9, 3.4 Hz, 1H), 4.64 (d, J=6.8 Hz, 1H), 3.46 (s, 3H), 3.36 (s, 3H), 2.00-1.86 (m, 1H), 1.85-1.74 (m, 1H), 1.48-1.35 (m, 2H), 1.34-1.24 (m, 4H), 0.86 (t, J=7.1 Hz, 3H).

¹³C NMR (101 MHz, CDCl₃) δ 166.1, 165.6, 133.3, 133.1, 130.3, 130.1, 130.0, 129.8, 128.6, 128.5, 102.1, 73.2, 72.3, 54.4, 53.7, 31.7, 29.4, 25.3, 22.6, 14.1.

ESI HRMS calculated for C₂₄H₃₀NaO₆ (M+Na)⁺: 437.1940, found 437.1943.

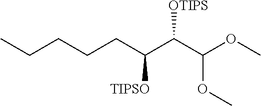

### (5S,6S)-5-(dimethoxymethyl)-3,3,8,8-tetraisopropyl-2,9-dimethyl-6-pentyl-4,7-dioxa-3,8-disiladecane (26)

(2S,3S)-1,1-dimethoxyoctane-2,3-diol (0.95 g, 4.61 mmol, 1.0 equiv.) was dissolved in 11.5 mL CH₂Cl₂ in a round-bottom flask charged with a stir bar, and the flask was sealed under Argon with stirring. Then, 2,6-lutidine (3.22 mL, 27.6 mmol, 6.0 equiv.) was added, followed by the dropwise addition of TIPSOTf (4.95 mL, 18.4 mmol, 4.0 equiv.). The resulting solution was stirred for 18 h and then quenched by the addition of 60 mL saturated NH₄Cl. The aqueous layer was extracted with Et₂O (3×40 mL) and the combined organic layers were washed with brine, dried over MgSO₄ and concentrated in vacuo. The resulting oil was purified using silica gel chromatography (0% to 5% Et₂O in hexanes, by volume) and the desired fractions concentrated (N.B. the product is readily stained using standard 2,4-dinitrophenylhydrazine TLC stain) in vacuo to give 1.92 g (80% yield) of 26 as a colourless oil.

¹H NMR (400 MHz, CDCl₃) δ 4.26 (d, J=6.6 Hz, 1H), 4.01-3.95 (m, 1H), 3.83 (dd, J=6.6, 1.4 Hz, 1H), 3.44 (s, 3H), 3.42 (s, 3H), 1.83-1.71 (m, 1H), 1.56-1.45 (m, 1H), 1.37-1.24 (m, 6H), 1.12-1.02 (m, 42H), 0.88 (t, J=6.9 Hz, 3H).

¹³C NMR (101 MHz, CDCl₃) δ 107.3, 77.3, 75.9, 56.3, 56.2, 34.3, 32.6, 26.0, 22.8, 18.5, 18.4, 18.4, 14.2, 13.0, 12.9.

DART HRMS calculated for C₂₈H₆₃O₄Si₂ (M+H)⁺: 519.4259 found 519.4269.

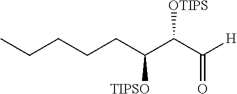

### (2S,3S)-2,3-bis((triisopropylsilyl)oxy)octanal (8)

26 (1.00 g, 1.93 mmol, 1.0 equiv.) was dissolved in 28.9 mL acetone in a round-bottom flask charged with a stir bar. To this was added p-TSA monohydrate (0.073 g, 0.39 mmol, 0.2 equiv.) and the flask was sealed. The reaction was warmed to 30° C. and stirred for 96 h, at which point NMR of an aliquot of the crude reaction demonstrated complete consumption of the starting material. The reaction was quenched with 3 mL of saturated NaHCO₃ and stirred for 10 minutes before filtering through a pad of sodium carbonate, washing with Et₂O. The eluent was concentrated in vacuo to give a yellow oil that was purified using silica gel chromatography (0% to 10% Et₂O in hexanes, by volume). Concentration of the desired fractions yielded 0.90 g (98% yield) of 8 as a colourless oil.

¹H NMR (400 MHz, CDCl₃) δ 9.68 (dd, J=2.2, 0.8 Hz, 1H), 4.11-4.05 (m, 1H), 4.03 (t, J=2.0 Hz, 1H), 1.80-1.69 (m, 1H), 1.57-1.47 (m, 1H), 1.33-1.20 (m, 6H), 1.10-1.00 (m, 42H), 0.88 (t, J=6.9 Hz, 3H).

¹³C NMR (101 MHz, CDCl₃) δ 204.9, 80.9, 78.0, 34.4, 32.1, 25.2, 22.7, 18.3, 18.2, 18.1, 18.1, 14.1, 12.7, 12.4.

DART HMRS calculated for C₂₆H₅₇O₃Si₂ (M+H)⁺: 473.3841 found 473.3844.

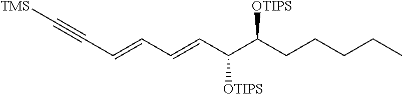

### (5S,6R)-3,3,8,8-tetraisopropyl-2,9-dimethyl-5-pentyl-6-((1E,3E)-6-(trimethylsilyl)hexa-1,3-dien-5-yn-1-yl)-4,7-dioxa-3,8-disiladecane (22)

9 (0.41 g, 1.68 mmol, 1.5 equiv.) was dissolved in 5.0 mL THF in a round-bottom flask charged with a stir bar. The flask was sealed under Argon and cooled to −78° C. in a dry-ice acetone bath with stirring. Then n-BuLi* (1.6 M in hexanes, 1.0 mL, 1.6 mmol, 1.45 equiv.) was added and the mixture was allowed to stir at −78° C. for 10 minutes before being removed from the cooling bath and stirring for another 30 minutes. The reaction was cooled back to −78° C. and 8 (0.53 g dissolved in 2.25 mL THF, 1.12 mmol, 1.0 equiv.) was added to the reaction mixture. The reaction was stirred for 18 h, gradually warming to room temperature. The reaction was quenched with 10 mL saturated NH₄Cl and transferred to a separatory funnel. The aqueous layer was extracted with Et₂O (3×15 mL) and the combined organic layers were washed with brine (2×10 mL), dried over MgSO₄, and concentrated in vacuo. The resulting resin was purified using silica gel chromatography (0% to 5% Et₂O in hexanes, by volume) yielding 0.56 grams (0.94 mmol, 84% yield) of 22 as an E/Z mixture. This was immediately dissolved in 10.8 mL benzene in a round-bottom flask charged with a stir bar, to which was added a crystal of iodine (2.4 mg, 0.0094 mmol, 0.01 equiv.) and the light purple reaction mixture was allowed to stir for 12 h (if necessary, another 1-2 mol % of iodine can be added if the solution loses its purple colour). The reaction was quenched with 1.0 mL saturated Na₂S₂O₃ and 4 mL of water. The aqueous phase was washed with Et₂O (3×5 mL) in a separatory funnel and the combined organic fractions were washed with brine, dried over MgSO₄, and concentrated in vacuo to give 0.54 g of 22 as a pale-yellow oil (81% from 8). *n-BuLi should be titrated before use.

¹H NMR (400 MHz, CDCl₃) δ 6.65 (dd, J=15.6, 10.7 Hz, 1H), 6.11 (dd, J=15.4, 10.8 Hz, 1H), 5.88 (dd, J=15.5, 7.8 Hz, 1H), 5.57 (d, J=15.7 Hz, 1H), 4.15 (dd, J=7.8, 3.0 Hz, 1H), 3.87-3.81 (m, 1H), 1.50-1.43 (m, 2H), 1.34-1.23 (m, 6H), 1.06-1.01 (m, 42H), 0.88 (t, J=7.1 Hz, 3H), 0.19 (s, 9H).

¹³C NMR (101 MHz, CDCl₃) δ 142.6, 137.9, 130.4, 110.4, 104.5, 96.9, 77.4, 76.8, 34.6, 32.2, 24.9, 22.6, 18.3, 18.3, 18.2, 18.1, 14.1, 12.8, 12.5, 0.0.

DART HRMS calculated for C₃₄H₆₉O₂Si₃ (M+H)⁺: 593.4600 found 593.4593.

### Preparation of Horner-Wadsworth-Emmons (HWE) Reagent (9)

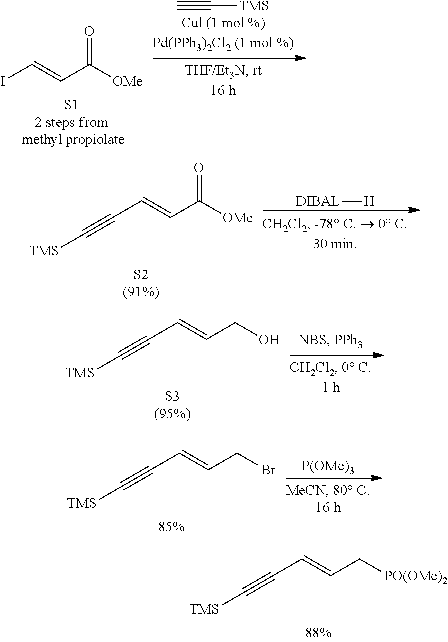

(E)-methyl-3-iodoacrylate is commercially available, but is also readily prepared in two steps from methyl propiolate.^{[2]}

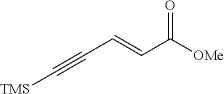 [SMILES: COC(=O)/C=C/C#C[Si](C)(C)C]

### (E)-methyl 5-(trimethylsilyl)pent-2-en-4-ynoate (S2)

(E)-methyl-3-iodoacrylate (3.78 g, 17.8 mmol, 1.0 equiv.) was dissolved in 35 mL THF in a round-bottom flask charged with a stir bar. The flask was purged with Argon gas prior to the addition of triethylamine (9.95 mL, 71.3 mmol, 4.0 equiv.), TMS-acetylene (2.92 mL, 20.5 mmol, 1.15 equiv.), PdCl₂(PPh₃)₂ (0.125 g, 0.178 mmol, 0.01 equiv.) and CuI (0.034 g, 0.178 mmol, 0.01 equiv.), in that order. The sealed reaction was stirred overnight during which period a significant amount of triethylamine hydrochloride had precipitated. The entire reaction mixture was filtered through a pad of silica, eluting with ether before concentrating in vacuo. The resulting oil was purified using silica gel chromatography (0% to 5% EtOAc in hexanes, by volume). After solvent removal, 2.95 g of the enyne 52 was obtained as a light-yellow oil (91% yield).

¹H NMR (400 MHz, CDCl₃) δ 6.74 (d, J=15.9 Hz, 1H), 6.24 (d, J=15.9 Hz, 1H), 3.75 (s, 3H), 0.21 (s, 9H).

¹³C NMR (101 MHz, CDCl₃) δ 166.4, 130.8, 125.3, 105.2, 101.4, 52.0, −0.3. All data are in agreement with the literature.^{[3]}

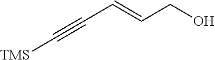

### (E)-5-(trimethylsilyl)pent-2-en-4-yn-1-ol (S3)

TMS-protected enyne (2.90 g, 15.9 mmol, 1.0 equiv.) was dissolved in 50 mL CH₂Cl₂ in a round-bottom flask charged with a stir bar and sealed under Argon. The reaction mixture was cooled to 0° C. prior to the addition of DIBAL-H (1.0 M in DCM, 37.9 mL, 37.9 mmol, 2.38 equiv.) over a 10-minute period; the reaction becomes dark golden but returns to near colourless once the addition is completed. The reaction was allowed to stir for another 1 h at 0° C. prior to the portion-wise addition of 50 mL water to quench, followed by 100 mL EtOAc. The resulting slurry was vacuum filtered through a fine sintered glass frit, with exhaustive rinsing of the gel-like precipitate with EtOAc (approx. 100 mL). The organic filtrate was then washed with water (50 mL) and brine (50 mL) in a separatory funnel before drying over Na₂SO₄ and concentrating in vacuo. The resulting oil was purified using silica gel chromatography (0% to 25% EtOAc in hexanes, by volume). After solvent removal, 2.34 g of the allylic alcohol S3 was obtained as a colourless oil (95% yield).

¹H NMR (400 MHz, CDCl₃) δ 6.30 (dt, J=16.0, 5.1 Hz, 1H), 5.76 (dt, J=16.0, 1.8 Hz, 1H), 4.19 (dd, J=5.1, 1.8 Hz, 2H), 1.65 (s, OH, 1H), 0.18 (s, 9H).

¹³C NMR (101 MHz, CDCl₃) δ 143.1, 110.4, 103.1, 95.4, 63.0, 0.0. All data are in agreement with the literature.^{[3]}

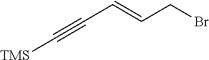

### (E)-(5-bromopent-3-en-1-yn-1-yl)trimethylsilane (54)

Allylic alcohol (2.34 g, 15.2 mmol, 1.0 equiv.) was dissolved in 30 mL CH₂Cl₂ in a two-neck round-bottom flask charged with a stir bar. Then, N-bromosuccinimide (3.51 g, 19.7 mmol, 1.3 equiv.) was added and the flask was sealed and placed under Argon atmosphere and cooled to 0° C., with stirring. Then PPh₃ (5.17 g, 19.7 mmol, 1.3 equiv.) was added portion-wise and the reaction was stirred for 1 h at 0° C., at which point the starting material had been consumed by TLC. The flask was concentrated in vacuo to give a thick orange oil that was then triturated with 20 mL of 1:1 hexanes/ether, resulting in the formation of an orange precipitate. The mixture was filtered, and the precipitate washed with 50 mL of 1:1 hexanes/ether. The filtrate was concentrated in vacuo and purified using silica gel chromatography (0% to 5% Et₂O in hexanes, by volume) to yield 2.80 g of the allylic bromide 54 as a light-yellow oil (85% yield) after solvent removal.

¹H NMR (400 MHz, CDCl₃) δ 6.30 (dt, J=15.6, 7.8 Hz, 1H), 5.75 (dt, J=15.6, 1.1 Hz, 1H), 3.97 (dd, J=7.8, 1.1 Hz, 2H), 0.19 (s, 9H).

¹³C NMR (101 MHz, CDCl₃) δ 139.1, 114.5, 102.1, 97.8, 31.6, −0.1. All data are in agreement with the literature.^{[4]}

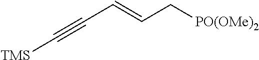

### (E)-dimethyl (5-(trimethylsilyl)pent-2-en-4-yn-1-yl)phosphonate (9)

Allylic bromide (1.50 g, 6.91 mmol, 1.0 equiv.) was dissolved in 7.50 mL MeCN and added to a cylindrical 100 mL pressure flask charged with a stir bar. Then, trimethyl phosphite (8.15 mL, 69.1 mmol, 10.0 equiv.) was added and the flask was sealed. The bottom portion of the flask containing the reaction mixture was heated to 80° C. on an aluminum heating block. The reaction was stirred with heating for 16 h, with pressure being relieved from the flask by briefly opening and resealing it at 0.5 h and 1.5 h post reaction initiation. After heating for 16 h, the reaction mixture was concentrated in vacuo to remove any volatiles, and the resulting oil was redissolved in 10 mL Et₂O and thoroughly washed with brine (3×10 mL) in a separatory funnel. The organic layer was dried over MgSO₄, concentrated in vacuo, and the resulting oil was purified using silica gel chromatography (50% to 100% EtOAc in hexanes, by volume). After solvent removal, 1.50 g of 9 was obtained as a colourless oil (88% yield).

¹H NMR (400 MHz, CDCl₃) δ 6.01 (dtd, J_{HH}=15.7, 7.6 Hz, ³J_{PH}=7.6 Hz, 1H), 5.56 (dtd, J_{HH}=15.7, 1.4 Hz, ⁴J_{PH}=5.4 Hz, 1H), 3.66 (d, ²J_{PH}=11.0 Hz, 6H), 2.58 (ddd, J_{HH}=7.6, 1.4 Hz, ²J_{PH}=22.8 Hz, 2H), 0.08 (s, 9H).

¹³C NMR (101 MHz, CDCl₃) δ 133.2 (d, ²J_{PC}=12.4 Hz), 114.8 (d, ³J_{PC}=15.8 Hz), 102.6 (d, ⁴J_{PC}=5.4 Hz), 95.0 (d, ⁵J_{PC}=3.0 Hz), 52.8 (d, ²J_{PC}=6.8 Hz), 30.1 (d, ¹J_{PC}=140 Hz), −0.2.

³¹P NMR (162 MHz, CDCl₃) δ 27.88.

DART HRMS calculated for C₁₀H₂₀O₃SiP (M+H)⁺: 247.0914 found 247.0916.

Preparation of vinyl iodide fragment (5)^{[5]}

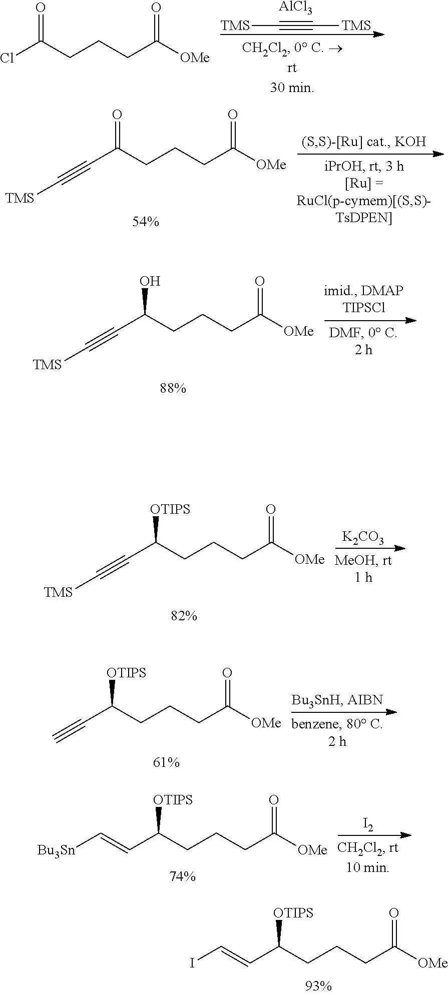

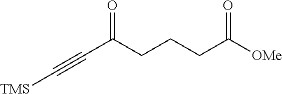 [SMILES: COC(=O)CCCC(=O)C#C[Si](C)(C)C]

### Methyl 5-oxo-7-(trimethylsilyl)hept-6-ynoate (56)

In a 100 mL round-bottom flask equipped with a stir bar was added AlCl₃ (2.11 g, 15.8 mmol, 1.3 equiv.) in CH₂Cl₂ (15 mL, 0.8 M) at room temperature under Argon atmosphere. The solution was cooled to 0° C. with an ice/water bath to which a solution of acid chloride 5S (1.68 mL, 12.2 mmol, 1.0 equiv.) and bis-TMS acetylene in CH₂Cl₂ (20 mL) was added over 10 minutes to the suspension at 0° C. The resulting yellow mixture was allowed to slowly warm to room temperature over 30 minutes. Upon completion as indicated by TLC analysis, the mixture was cooled back down to 0° C. and quenched by the slow addition of 1N HCl (15 mL). The two phases were separated, and the aqueous layer was extracted twice with CH₂Cl₂. The combined organic layers was then washed with brine, dried over Na₂SO₄, filtered, and concentrated in vacuo. The crude residue was purified by flash column chromatography on silica gel (hexanes/ethyl acetate 9:1) to afford the alkynone 56 (1.48 g, 54% yield) as a pale-yellow oil.

¹H NMR (300 MHz, CDCl₃) δ 3.67 (t, J=1.0 Hz, 3H), 2.65 (td, J=7.2, 1.2 Hz, 2H), 2.36 (dd, J=8.0, 6.6 Hz, 2H), 2.07-1.89 (m, 2H), 0.23 (t, J=1.0 Hz, 9H). All data are in agreement with the literature.^{[5]}

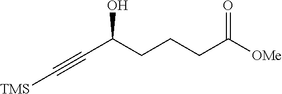 [SMILES: COC(=O)CCC[C@H](O)C#C[Si](C)(C)C]

### Methyl (S)-5-hydroxy-7-(trimethylsilyl)hept-6-ynoate (57)

In a 100 mL round-bottom flask equipped with a stir bar was added RuCl(p-cymene)[(S,S)-TsDPEN](208 mg, 0.33 mmol, 5 mol %) and potassium hydroxide (184 mg, 3.27 mmol, 0.5 equiv.) dissolved in CH₂Cl₂ (9 mL). The mixture was allowed to stir at room temperature for 5 minutes, then washed with water six times. The CH₂Cl₂ layer was dried over Na₂SO₄, filtered, and concentrated in vacuo to yield a purple solid. The purple solid catalyst was then redissolved in iPrOH (9 mL) at room temperature, to which a solution of alkynone (1.48 g, 6.54 mmol, 1.0 equiv.) in iPrOH was added. The resulting mixture was allowed to stir at room temperature for 3 h. Upon completion as indicated by TLC analysis, the solvent was removed in vacuo and the crude residue was loaded directly on a silica gel column for purification. The crude residue was purified by flash column chromatography on silica gel (hexanes/ethyl acetate 9:1) to furnish the chiral propargyl alcohol S7 (1.31 g, 88% yield) as a pale-yellow oil.

¹H NMR (300 MHz, CDCl₃) δ 4.37 (q, J=5.7 Hz, 1H), 3.68 (d, J=0.8 Hz, 3H), 2.38 (t, J=6.9 Hz, 2H), 1.91 (d, J=5.3 Hz, 1H), 1.88-1.66 (m, 3H), 0.16 (d, J=0.8 Hz, 9H). All data are in agreement with the literature.^{[5]}

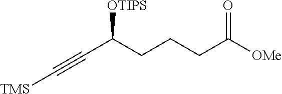

### Methyl (S)-5-((triisopropylsilyl)oxy)-7-(trimethylsilyl)hept-6-ynoate (58)

In a 100 mL round-bottom flask equipped with a stir bar was added propargyl alcohol S7 (1.31 g, 5.74 mmol, 1.0 equiv.) dissolved in DMF (6 mL, 1 M) at room temperature under Argon atmosphere. The solution was then cooled to 0° C. in an ice/water bath, to which imidazole (1.17 g, 17.2 mmol, 3.0 equiv.), DMAP (35 mg, 0.3 mmol, 5 mol %), and TIPSCl (2.46 mL, 11.5 mmol, 2.0 equiv.) were added sequentially at 0° C. The mixture was allowed to stir at 0° C. for 2 h. Upon completion as indicated by TLC analysis, the reaction mixture was quenched by the addition of water (10 mL) and diluted with Et₂O. The aqueous layer was extracted with Et₂O twice. The combined organic layers were washed again with water and brine, dried over Na₂SO₄, filtered, and concentrated in vacuo. The crude residue was purified by flash column chromatography on silica gel (hexanes/ethyl acetate 19:1) to afford the silyl ether 58 (1.8 g, 82% yield) as a pale-yellow oil.

¹H NMR (300 MHz, CDCl₃) δ 4.53-4.42 (m, 1H), 3.67 (d, J=2.2 Hz, 3H), 2.42-2.31 (m, 2H), 1.88-1.64 (m, 4H), 1.22-1.01 (m, 21H), 0.14 (d, J=2.2 Hz, 9H). All data are in agreement with the literature.^{[5]}

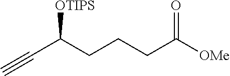

### Methyl (S)-5-((triisopropylsilyl)oxy)hept-6-ynoate (59)

In a 100 mL round-bottom flask equipped with a stir bar was added TIPS-protected propargyl alcohol S8 (1.8 g, 4.6 mmol, 1.0 equiv.) dissolved in HPLC-grade methanol (46 mL, 0.1 M) at room temperature. Potassium carbonate (636 mg, 4.6 mmol, 1.0 equiv.) was added to the reaction solution in one portion at room temperature. The resulting mixture was allowed to stir at room temperature for 4 h. Upon completion as indicated by TLC analysis, the solvent was removed in vacuo. The residue was redissolved in ethyl acetate and water. The two phases were separated, and the aqueous layer was extracted with ethyl acetate twice. The combined organic layers were dried over Na₂SO₄, filtered, concentrated in vacuo. The crude residue was purified by flash column chromatography on silica gel (hexanes/ethyl acetate 19:1) to afford the terminal alkyne 59 (875 mg, 61% yield) as a pale-yellow oil.

¹H NMR (300 MHz, CDCl₃) δ 4.50 (td, J=5.8, 2.1 Hz, 1H), 3.67 (s, 3H), 2.42-2.32 (m, 2H), 1.92-1.66 (m, 4H), 1.31-0.92 (m, 21H). All data are in agreement with the literature.^{[5]}

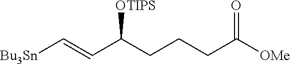

### Methyl (S,E)-7-(tributylstannyl)-5-((triisopropylsilyl)oxy)hept-6-enoate (510)

In a 100 mL round-bottom flask equipped with a stir bar was added terminal alkyne 59 (560 mg, 1.8 mmol, 1.0 equiv.) dissolved in benzene (35 mL, 0.05 M) at room temperature under Argon atmosphere. AIBN (60 mg, 0.36 mmol, 0.2 equiv.) and tributyltin hydride (1.5 mL, 5.4 mmol, 3.0 equiv.) were added sequentially to the reaction flask at room temperature. The resulting mixture was heated to 80° C. on an aluminum heating block and allowed to stir at 80° C. for 2 h. Upon completion as indicated by TLC analysis, the reaction mixture was allowed to cool down to room temperature where the solvent was removed in vacuo. The crude residue was loaded directly onto silica gel (hexanes/ethyl acetate 19:1) for purification by flash column chromatography to afford vinyl stannane 510 (800 mg, 74% yield) as a pale-yellow oil.

¹H NMR (300 MHz, CDCl₃) δ 6.13-5.78 (m, 2H), 4.14 (p, J=6.9, 6.2 Hz, 1H), 3.69-3.62 (m, 3H), 2.31 (t, J=7.2 Hz, 2H), 1.53-1.40 (m, 13H), 1.35-1.17 (m, 9H), 1.05 (d, J=1.9 Hz, 18H), 0.95-0.80 (m, 9H). All data are in agreement with the literature.^{[5]}

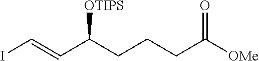

### Methyl (S,E)-7-iodo-5-((triisopropylsilyl)oxy)hept-6-enoate (5)

In a 100 mL round-bottom flask equipped with a stir bar was added vinyl stannane 510 (800 mg, 1.3 mmol, 1.0 equiv.) dissolved in CH₂Cl₂ (7 mL) at room temperature. A solution of I₂ (505 mg, 2.0 mmol, 1.5 equiv.) in CH₂Cl₂ (10 mL) was added dropwise until the resulting mixture remained a light pink colour. The mixture was then allowed to stir at room temperature for 10 minutes until completion as indicated by TLC analysis. The reaction was quenched by the addition of saturated Na₂S₂O₃, water, and saturated NaHCO₃. The biphasic mixture was allowed to stir at room temperature for an additional 5 minutes. The phases were then separated, and the aqueous layer was extracted three times with CH₂Cl₂. The combined organic layers were dried over Na₂SO₄, filtered, and concentrated in vacuo. The crude residue was purified by flash column chromatography on silica gel (hexanes/ethyl acetate 19:1) to afford vinyl iodide fragment 5 (543 mg, 93% yield) as a pale-yellow oil.

¹H NMR (300 MHz, CDCl₃) δ 6.58-6.45 (m, 1H), 6.24 (dt, J=14.5, 1.3 Hz, 1H), 4.23 (q, J=5.7 Hz, 1H), 3.67 (d, J=1.2 Hz, 3H), 2.32 (t, J=7.0 Hz, 2H), 1.74-1.48 (m, 7H), 1.04 (t, J=1.8 Hz, 18H). All data are in agreement with the literature.^{[5]}

### Procedure for the Final Steps

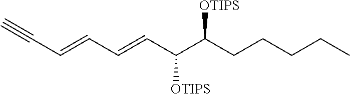

### (5R,6S)-5-((1E,3E)-hexa-1,3-dien-5-yn-1-yl)-3,3,8,8-tetraisopropyl-2,9-dimethyl-6-pentyl-4,7-dioxa-3,8-disiladecane (4)

22 (0.54 g, 0.91 mmol, 1.0 equiv.) was dissolved in 2.4 mL CH₂Cl₂ and added to 12 mL MeOH in a round-bottom flask charged with a stir bar. Then, potassium carbonate (0.57 g, 4.1 mmol, 4.5 equiv.) was added and the mixture was stirred vigorously for 2 h at room temperature. Then, 10 mL water and 10 mL Et₂O were added, and the aqueous phase was extracted with Et₂O (3×15 mL). The organic layers were combined and washed with brine (2×10 mL), dried over MgSO₄, and concentrated in vacuo to yield 0.46 g (97% yield) of 4 as a pale-yellow oil that was used without further purification.

¹H NMR (400 MHz, CDCl₃) δ 6.67 (dd, J=15.7, 10.7 Hz, 1H), 6.13 (dd, J=15.5, 10.8 Hz, 1H), 5.89 (dd, J=15.5, 7.7 Hz, 1H), 5.54 (dd, J=15.7, 2.4 Hz, 1H), 4.16 (dd, J=7.7, 2.9 Hz, 1H), 3.84 (ddd, J=6.7, 5.1, 3.3 Hz, 1H), 3.02 (d, J=2.4 Hz, 1H), 1.53-1.45 (m, 2H), 1.36-1.27 (m, 6H), 1.05-1.04 (m, 22H), 1.04-1.03 (m, 20H), 0.88 (t, J=7.0 Hz, 3H).

¹³C NMR (101 MHz, CDCl₃) δ 143.4, 138.4, 130.2, 109.4, 83.1, 79.5, 77.5, 76.8, 34.7, 32.4, 25.0, 22.8, 18.4, 18.4, 18.3, 18.2, 14.2, 13.0, 12.7.

DART HRMS calculated for C₃₁H₆₁O₂Si₂ (M+H)⁺: 521.4205 found 521.4213.

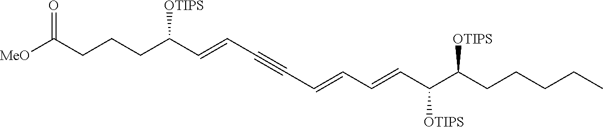

### Methyl (5S,6E,10E,12E,14R,15S)-5,14,15-tris((triisopropylsilyl)oxy)octadeca-6,10,12-dien-8-ynoate (27)

In a 20 mL scintillation vial equipped with a stir bar was added vinyl iodide fragment 5 (217 mg, 0.494 mmol, 1.0 equiv.) and terminal alkyne fragment 4 (283 mg, 0.543 mmol, 1.1 equiv.) dissolved in degassed tert-butylamine (2 mL) at room temperature under Ar atmosphere. The headspace was evacuated and charged with Ar three times. Pd(PPh₃)₄ (28 mg, 0.025 mmol, 5 mol %) and CuI (9.4 mg, 0.049 mmol, 10 mol %) were then added to the mixture sequentially at room temperature. The resulting mixture was allowed to stir at room temperature for 20 minutes until completion as indicated by TLC analysis. Upon completion, saturated NH₄Cl was added to the mixture and diluted with ethyl acetate. After separation, the aqueous layer was extracted with ethyl acetate twice. The combined organic layers were dried over Na₂SO₄, filtered, and concentrated in vacuo. The crude residue was purified by flash column chromatography on silica gel (hexanes/ethyl acetate 9:1) to afford the coupled trienyne (420 mg, 93% yield) as a yellow oil.

¹H NMR (400 MHz, CDCl₃) δ 6.58 (dd, J=15.5, 10.8 Hz, 1H), 6.18-6.07 (m, 2H), 5.90-5.79 (m, 2H), 5.67 (dd, J=15.6, 2.4 Hz, 1H), 4.35 (q, J=5.6 Hz, 1H), 4.15 (dd, J=7.9, 3.3 Hz, 1H), 3.86-3.81 (m, 1H), 3.66 (s, 3H), 2.33-2.28 (m, 2H), 1.74-1.44 (m, 8H), 1.34-1.21 (m, 7H), 1.07-1.03 (m, 63H), 0.87 (t, J=7.2 Hz, 3H).

¹³C NMR (75 MHz, CDCl₃) δ 174.0, 145.8, 141.4, 137.5, 130.8, 110.7, 109.6, 90.5, 89.5, 77.5, 76.9, 72.5, 51.6, 37.5, 34.7, 34.2, 32.4, 25.0, 22.7, 19.9, 18.4, 18.4, 18.3, 18.2, 14.1, 13.0, 12.7, 12.5.

DART HRMS calculated for C₄₈H₉₂O₅SiNa (M+Na)⁺: 855.6150 found 855.6143.

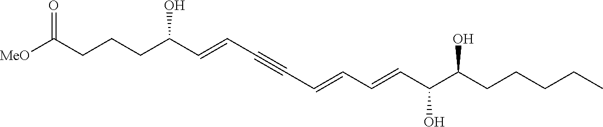 [SMILES: CCCCC[C@H](O)[C@H](O)/C=C/C=C/C#C/C=C/[C@@H](O)CCCC(=O)OC]

### Methyl (5S,6E,10E,12E,14R,15S)-5,14,15-trihydroxyicosa-6,10-12-trien-8-ynoate (28)

In a 20 mL scintillation vial equipped with a stir bar was added bis-silyated trienyne (140 mg, 0.21 mmol, 1.0 equiv.) dissolved in anhydrous THF (10 mL) at room temperature under Ar atmosphere. TBAF (1 M in THF, 0.52 mL, 0.52 mmol, 2.5 equiv.) was added dropwise to the reaction solution at room temperature under Ar atmosphere. The resulting mixture was allowed to stir at room temperature for 2 h. Upon completion as indicated by TLC analysis, the solvent was removed in vacuo followed by its dilution with diethyl ether and saturated NH₄Cl. After separation, the aqueous layer was extracted twice with diethyl ether. The combined organic layers were dried over Na₂SO₄, filtered, and concentrated in vacuo. The crude residue was purified by flash column chromatography on silica gel (hexanes/ethyl acetate 1:1) to afford the deprotected triol (61 mg, 81% yield) as a yellow oil.

¹H NMR (400 MHz, CDCl₃) δ 6.57 (dd, J=15.4, 10.9 Hz, 1H), 6.32 (dd, J=15.3, 10.8 Hz, 1H), 6.11 (dd, J=15.8, 6.1 Hz, 1H), 5.89-5.79 (m, 2H), 5.73 (dd, J=15.5, 2.3 Hz, 1H), 4.21-4.11 (m, 2H), 3.72-3.67 (m, 1H), 3.66 (s, 3H), 2.47 (br s, 3H), 2.34 (t, J=7.3 Hz, 2H), 1.78-1.64 (m, 2H), 1.59-1.53 (m, 2H), 1.43-1.24 (m, 8H), 0.87 (d, J=6.4 Hz, 3H).

¹³C NMR (101 MHz, CDCl₃) δ 174.298, 145.279, 140.779, 133.649, 132.007, 112.041, 110.249, 90.665, 89.696, 75.331, 74.500, 71.753, 51.765, 36.190, 33.763, 32.123, 31.882, 25.647, 22.663, 20.716, 14.137.

ESI HRMS calculated for C₂₁H₃₂O₅Na (M+Na)⁺: 387.2147 found 387.2162.

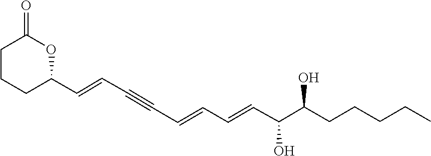 [SMILES: CCCCC[C@H](O)[C@H](O)/C=C/C=C/C#C/C=C/[C@@H]1CCCC(=O)O1]

### (S)-6-((1E,5E,7E,9R,105)-9,10-dihydroxypentadeca-1,5,7-trien-3-yn-1-yl)tetrahydro-2H-pyran-2-one (29)[6]

In a 100 mL round bottom flask equipped with a stir bar was added bis-silyated trienyne (375 mg, 0.55 mmol, 1.0 equiv.) dissolved in anhydrous THF (28 mL) at room temperature under Ar atmosphere. TBAF (1 M in THF, 1.4 mL, 1.39 mmol, 2.5 equiv.) was added dropwise to the reaction solution at room temperature under Ar atmosphere. The resulting mixture was allowed to stir at room temperature for 2 h. Upon completion as indicated by TLC analysis, the solvent was removed in vacuo followed by its dilution with diethyl ether and saturated NH₄Cl. After separation, the aqueous layer was extracted twice with diethyl ether. The combined organic layers were dried over Na₂SO₄, filtered, and concentrated in vacuo. The crude residue was purified by flash column chromatography on silica gel (hexanes/ethyl acetate 1:1) to afford the lactone (9 mg, 10% yield) as a yellow oil. All characterization data are in agreement with the literature.^{[6]}

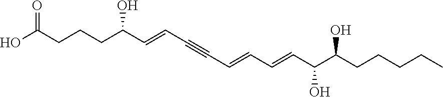 [SMILES: CCCCC[C@H](O)[C@H](O)/C=C/C=C/C#C/C=C/[C@@H](O)CCCC(=O)O]

### (5S,6E,10E,12E,14R,15S)-5,14,15-trihydroxyicosa-6,10,12-trien-8-ynoic acid (3)

In a 20 mL scintillation vial equipped with a stir bar was added the lactone (82 mg, 0.25 mmol, 1.0 equiv.) or triene methyl ester (50 mg, 0.14 mmol, 1.0 equiv.) dissolved in a 3:1 mixture of THF/H₂O (0.2M). LiOH (1.2 equiv.) was added in one portion to the solution at room temperature. The resulting mixture was allowed to stir at room temperature for 3 hours or overnight. Upon completion, the reaction mixture was filtered to remove any residual LiOH solid and washed with THF. The remaining solvent was removed in vacuo to afford a sticky crude residue, which was triturated with diethyl ether three times. The solid was filtered and washed with diethyl ether to afford the carboxylic acid in quantitative yield as a white solid.

¹H NMR (300 MHz, MeOD) δ 6.57 (dd, J=15.3, 10.8 Hz, 1H), 6.35 (dd, J=15.1, 10.9 Hz, 1H), 6.09 (dd, J=15.7, 6.0 Hz, 1H), 5.96-5.70 (m, 3H), 4.12 (q, J=6.1 Hz, 1H), 3.96 (t, J=5.9 Hz, 1H), 3.53-3.44 (m, 1H), 2.18 (t, J=7.2 Hz, 2H), 1.74-1.48 (m, 6H), 1.33 (d, J=0.0 Hz, 6H), 0.91 (t, J=6.3 Hz, 3H).

¹³C NMR (75 MHz, MeOD) δ 182.665, 147.088, 142.172, 136.913, 132.103, 112.250, 110.432, 91.295, 89.964, 76.512, 75.660, 72.576, 38.826, 37.979, 33.782, 33.072, 26.628, 23.721, 23.539, 14.436.

ESI HRMS calculated for C₂₀H₂₉O₅(M−H)⁻: 349.2020, found 349.2023.

### Preparation of LXB₄Via the Karstedt Alkyne Hydrosilylation/Proto-Desilylation Protocol

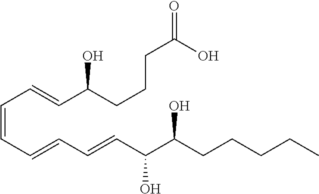 [SMILES: CCCCC[C@H](O)[C@H](O)/C=C/C=C/C=C\C=C\[C@@H](O)CCCC(=O)O]

### (5S,6E,8Z,10E,12E,14R,15)-5,14,15-trihydroxyicosa-6,8,10,12-tetraenoic acid (LXB₄) (2)

Globally protected acetylene 27 (420 mg, 0.504 mmol, 1.0 equiv.) was dissolved in a solution of anhydrous toluene (4.0 mL) under argon. Dimethylethoxysilane (560 uL, 4.03 mmol, 8.0 equiv.) was added in one portion, followed by dropwise addition of Karstedt's catalyst (platinum(0)-1,3-divinyl-1,1,3,3,-tetramethyldisoloxane complex, 2% Pt) (115 μL, 0.010 mmol, 2 mol %). The round bottom flask was covered with foil to prevent UV exposure and the solution was stirred overnight at room temperature. After approximately 16 hours, TLC analysis showed complete conversion of the starting material (R_{f}=0.8 in hexanes/Et₂O 9:1) and the appearance of two new spots (R_{f}=0.65 and 0.7), which were assumed to be the two isomers generated by the hydrosilylation across the triple bond. The crude reaction mixture was filtered through a silica plug, which was washed with Et₂O, and the collected filtrate was dried under vacuum to afford a yellow-brown oil. This oil was redissolved in anhydrous THF (4.4 mL) under argon. The solution was again wrapped in foil, and a solution of TBAF in THF (1.0 M, 4.0 mL, 8.0 equiv.) was added dropwise. The reaction was stirred at room temperature for 20 minutes. TLC showed residual silylated material (R_{f}=0.9 in CH₂Cl₂/Et₂O/MeOH 1:1:0.05) in addition to the desired methyl ester (R_{f}=0.35) and lactone (R_{f}=0.3). Another 3 equiv. of TBAF (300 μL) was added dropwise. After another 20 minutes, TLC showed complete conversion to the methyl ester and lactone. The reaction was quenched by the addition of phosphate buffer (pH 7) and water and extracted with diethyl ether. The combined organic phase was dried over Na₂SO₄ and concentrated in vacuo. The crude oil was immediately redissolved in THF/H₂O (3:1, 2.5 mL) and treated with LiOH (12.1 mg, 0.50 mmol, 1.0 equiv.) mmol. The resulting solution was stirred at room temperature in a round-bottom flask wrapped in aluminium foil until TLC indicated complete conversion. The reaction mixture was flash filtered through celite to remove any residual LiOH solid and washed with THF. The remaining solvent was removed in vacuo to afford a sticky yellow residue, which was immediately redissolved in MeOH. Storage of the crude or purified material as a solid at room temperature resulted in rapid degradation, and stability was greatly improved by storing the material as a methanolic solution. This material was purified by reverse phase HPLC (XBridge BEH OBD Prep column, 130A, 5 μM, 30 mm×150 mm) using a mobile phase of H₂O+0.05% NH₄OH (A) and acetonitrile+0.05% NH₄OH (B) and a gradient elution of 97:3 A:B to 67:33 A:B. After lyophilization, we obtained 98.7 mg of LXB₄ for a yield of 56% over three steps. LXB₄ should be stored on dry ice until injected into the HPLC. In a solution in methanol at room temperature, formation of impurities which are extremely difficult to separate by HPLC occurred in 1-2 hours. LXB₄ was found to be stable in HPLC buffer for several hours.

¹H NMR (700 MHz, MeOD) δ 6.80-6.70 (m, 2H), 6.38 (dd, J=15.3, 10.8 Hz, 1H), 6.28 (dd, J=14.7, 10.9 Hz, 1H), 6.04-5.96 (m, 2H), 5.83 (dd, J=15.2, 7.1 Hz, 1H), 5.73 (dd, J=15.0, 6.6 Hz, 1H), 4.64 (br s, 1H), 4.16 (q, J=5.9 Hz, 1H), 3.97 (ddd, J=7.1, 5.0, 1.2 Hz, 1H), 3.51-3.47 (m, 1H), 2.19 (t, J=7.4 Hz, 2H), 1.72-1.51 (m, 6H), 1.38-1.27 (m, 6H), 0.91 (t, J=7.1 Hz, 3H).

¹³C NMR (176 MHz, MeOD) δ 182.815, 138.882, 134.672, 134.383, 133.392, 130.381, 130.293, 129.214, 126.440, 76.859, 75.727, 73.028, 38.753, 38.361, 33.801, 33.092, 26.647, 23.730, 23.603, 14.426.

HPLC (Acetonitrile+0.1% TFA, MilliQ water+0.1% TFA, gradient from 10:90→95:5): retention time 9.97 min (for commercial sample, retention time 9.95 min), purity 97%

ESI HRMS calculated for C₂₀H₃₂O₅Na [M+Na]*, predicted m/z 375.2147, found 375.2152.

[α]D₂₀=+9.2° (MeOH, c 0.001)

### Measurement of LXB₄ In Vitro Bioactivity in a Neuronal Injury Model

### HT22 Cell Maintenance

HT22 cells (SCC129, EMD Millipore) were grown on 100-mm tissue culture dishes (83.3902.300, Sarstedt) and maintained in high glucose DMEM (Sigma, D5796-500ML) supplemented with 10% fetal bovine serum (080-150, Wisent) and 1% Penstrep (15140122, Gibco) at 37° C. in a 5% CO₂ atmosphere incubator (67/340,1045, CellXpert C170, Eppendorf). Cells used for experiments were maintained at a <80% confluency and <25 passages, and were dissociated using TrypIE (12604013, Gibco).

### Treatment and Metabolic Stress

HT22 cells were seeded at 4×10³ cells in 100 μL of the high glucose DMEM+10% fetal bovine serum+1% Penstrep media on replicate wells of 96-well plates (83.3924.300, Sarstedt) for 20 hours at 37° C. in a 5% CO₂ atmosphere humidified incubator. Cells were then pretreated with 5 μL of the indicated LXB₄ concentration, or vehicle, one hour prior to addition of 3 mM glutamate stressor. Cells were exposed for 13 hours, then evaluated for cell viability using an XTT assay, according to the manufacturer's instructions (11465015001, Roche). The resulting absorbance values were read in a BMG CLARIOstar microplate reader under 490 nM excitation following 2 hours of XTT incubation and analyzed using Graphpad Prism 9 for ANOVA analyses and curve fitting.

Preparation of [³H]-LXB₄Via Semi-Hydrogenation with Tritium Gas

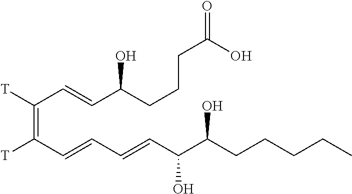

### (5S,6E,8Z,10E,12E,14R,15)-5,14,15-trihydroxyicosa-6,8,10,12-tetraenoic-8,9-t₂ acid ([³H]-LXB₄) (2T)

In a 50 mL round bottom flask equipped with a stir bar was added Lindlar's catalyst (25 mg, 30 wt %) in HPLC-grade methanol (3 mL, 0.1M) at room temperature. The headspace was evacuated and charged with tritium gas three consecutive times. A mixture of trienyne (0.25 mmol, 1.0 equiv.) and quinoline* (0.3 mL, 0.25 mmol, 1.0 equiv.) in HPLC-grade methanol (3 mL) was added to the suspension at room temperature under tritium gas atmosphere. The resulting mixture was allowed to stir at room temperature for 3 hours. Upon completion, the reaction mixture was filtered through a pad of Celite and washed with methanol. The remaining solvent was removed in vacuo to afford an oily crude residue. The residue was purified by reverse-phase HPLC (XBridge C18, 5 μm, 4.6×250 mm) using a mobile phase of H₂O+0.1% trifluoroacetic acid (A) and acetonitrile+0.1% trifluoroacetic acid (B) and a gradient elution of 65:35 A:B for 30 min at 22° C.; t_{R} (min.)=14.92. *Quinoline should be distilled before use.

HPLC (Acetonitrile+0.1% TFA, water+0.1% TFA, isocratic gradient at 35:65): retention time 14.92 min., radiochemical purity 96%

ESI HRMS calculated for C₂₀H₂₉T₂O₅ [M−H]⁻, found m/z 355.2369.

### REFERENCES

[1] C. N. Serhan, M. Hamberg, B. Samuelsson, Biochem. Biophys. Res. Commun. 1984, 118, 943-949.
[2] C. N. Serhan, M. Hamberg, B. Samuelsson, Proc. Natl. Acad. U.S.A. 1984, 81, 5335-5339.
[3] J. Adams, B. J. Fitzsimmons, Y. Girard, Y. Leblanc, J. F. Evans, J. Rokach, J. Am. Chem. Soc. 1985, 107, 464-469.
[4] C. N. Serhan, M. Hamberg, B. Samuelsson, J. Morris, D. G. Wishka, Proc. Natl. Acad. Sci. U.S.A. 1986, 83, 1983-1987.
[5] C. N. Serhan, K. C. Nicolaou, S. E. Webber, C. A. Veale, S. E. Dahlén, T. J. Puustinen, B. Samuelsson, J. Biol. Chem. 1986, 261, 16340-16345.
[6] For the role of lipoxins in inflammation, see a) J. Pirault, M. Bäck, Front. Pharmacol. 2018, 9, 1273; b) J. A. Chandrasekharan, N. Sharma-Wali, J. Inflamm. Res. 2015, 8, 181-192; c) M. Romano, E. Cianci, F. Simiele, A. Recchiuti, Eur. J. Pharmacol. 2015, 760, 49-63; d) C. N. Serhan, N. Chiang, J. Dalli, B. D. Levy, Cold Spring Harb. Perspect. Biol. 2015, 7, a016311; e) C. N. Serhan, Nature 2014, 510, 92-101; f) B. D. Levy, C. N. Serhan, Annu. Rev. Physiol. 2014, 76, 467-492; g) C. D. Russell, J. Schwarze, Immunology 2014, 141, 166-173; h) A. Recchiuti, C. N. Serhan, Front. Immunol. 2012, 3, 1-23; i) C. N. Serhan, Am. J. Pathol. 2010, 177, 1576-1591; j) C. D. Duffy, P. J. Guiry, Medchemcomm 2010, 1, 249-265; k) C. I. Svensson, M. Zattoni, C. N. Serhan, J. Exp. Med. 2007, 204, 245-252; l) J. F Parkinson, Inflamm. Allergy—Drug Targets 2006, 5, 91-106; m) C. N. Serhan, Prostaglandins Leukot. Essent. Fat. Acids 2005, 73, 141-162; n) B. McMahon, C. Godson, Am. J. Physiol.—Ren. Physiol. 2004, 286, 189-201; o) A. Kantarci, T. E. Van Dyke, Crit. Rev. Oral Biol. Med. 2003, 14, 4-12; p) C. Godson, S. Mitchell, K. Harvey, N. A. Petasis, N. Hogg, H. R. Brady, J. Immunol. 2000, 164, 1663-1667.
[7] F. S. Machado, J. E. Johndrow, L. Esper, A. Dias, A. Bafica, C. N. Serhan, J. Aliberti, Nat. Med. 2006, 12, 330-334.
[8] S. Fiore, J. F. Maddox, H. D. Perez, C. N. Serhan, J. Exp. Med. 1994, 180, 253-260.
[9] T. Takano, C. B. Clish, K. Gronert, N. Petasis, C. N. Serhan, J. Clin. Invest. 1998, 101, 819-826.
[10] M. Romano, J. F. Maddox, C. N. Serhan, J. Immunol. 1996, 157, 2149-2154.
[11] J. F. Maddox, C. N. Serhan, J. Exp. Med. 1996, 183, 137-146.
[12] For previous formal and total syntheses of LXB₄, see a) Y. Leblanc, B. Fitzsimmons, J. Adams, J. Rokach, Tetrahedron Lett. 1985, 26, 1399-1402; b) J. Morris, D. G. Wishka, Tetrahedron Lett. 1986, 27, 803-806; c) K. C. Nicolaou, S. E. Webber, Synthesis (Stuttg). 1986, 6, 453-461; d) A. Rodriguez, M. Nomen, B. W. Spur, J. J. Godfroid, T. H. Lee, Tetrahedron Lett. 2000, 41, 823-826; e) C. Gravier-Pelletier, J. Dumas, Y. Le Merrer, J. C. Depezay, Tetrahedron 1992, 48, 2441-2452; f) C. Gravier-Pelletier, J. Dumas, Y. Le Merrer, J. C. Depezay, Tetrahedron Lett. 1991, 32, 1165-1168; g) K. C. Nicolaou, B. E. Marron, C. A. Veale, S. E. Webber, C. N. Serhan, J. Org. Chem. 1989, 54, 5527-5535; h) M. Alami, B. Crousse, G. Linstrumelle, L. Mambu, M. Larchevêque, Tetrahedron Asymmetry 1997, 8, 2949-2958; i) K. C. Nicolaou, J. Y. Ramphal, N. A. Petasis, C. N. Serhan, Angew. Chemie Int. Ed. English 1991, 30, 1100-1116.
[13] C. Kim, I. Livne-Bar, K. Gronert, J. M. Sivak, Mol. Nutr. Food Res. 2020, 64, 1801076.
[14] I. Livne-Bar, J. Wei, H. H. Liu, S. Alqawlaq, G. J. Won, A. Tuccitto, K. Gronert, J. G. Flanagan, J. M. Sivak, J. Clin. Invest. 2017, 127, 4403-4414.
[15] R. E. Boer, J. A. Gimenez-Bastida, O. Boutaud, S. Jana, C. Schneider, G. A. Sulikowski, Org. Lett. 2018, 20, 4020-4022.
[16] V. S. Martin, S. S. Woodard, T. Katsuki, Y. Yamada, M. Ikeda, K. B. Sharpless, J. Am. Chem. Soc. 1981, 103, 6237-6240.
[17] Y. Suganuma, S. Tanabe, Y. Sugihara, Y. Kobayashi, Tetrahedron 2018, 74, 1151-1159.
[18] M. Morita, S. Wu, Y. Kobayashi, Org. Biomol. Chem. 2019, 17, 2212-2222.
[19] M. Morita, Y. Kobayashi, J. Org. Chem. 2018, 83, 3906-3914.
[20] For previous syntheses of related triene and tetraene natural products, see a) K. C. Nicolaou, S. E. Webber, J. Ramphal, Y. Abe, Angew. Chemie 1987, 99, 1077-1079; b) K. C. Nicolaou, C. A. Veale, S. E. Webber, H. Katerinopoulos, J. Am. Chem. Soc. 1985, 107, 7515-7518; c) K. Chauhan, R. K. Bhatt, J. R. Falck, J. H. Capdevila, Tetrahedron Lett. 1994, 35, 1825-1828; d) M. Nazarê, H. Waldmann, Tetrahedron Lett. 2000, 41, 625-628; e) D. Urabe, H. Todoroki, K. Masuda, M. Inoue, Tetrahedron 2012, 68, 3210-3219; f) E. D. Phillips, H. F. Chang, C. R. Holmquist, J. P. McCauley, Bloorganic Med. Chem. Lett. 2003, 13, 3223-3226; g) M. Nazarê, H. Waldmann, Chem. Eur. J. 2001, 7, 3363-3376; h) W. J. Guilford, J. G. Bauman, W. Skuballa, S. Bauer, G. P. Wei, D. Davey, C. Schaefer, C. Mallari, J. Terkelsen, J. L. Tseng, J. Shen, B Subramanyam, A. J. Schottelius, J. F. Parkinson, J. Med. Chem. 2004, 47, 2157-2165; i) K. C. Nicolaou, S. E. Webber, J. Chem. Soc. Chem. Commun. 1985, 5, 297-298; j) A. R. Rodriguez, B. W. Spur, Tetrahedron Lett. 2001, 42, 6057-6060; k) A. R. Rodriguez, B. W. Spur, Tetrahedron Lett. 2012, 53, 6990-6994; l) A. R. Rodriguez, B. W. Spur, Tetrahedron Lett. 2004, 45, 8717-8720; m) A. R. Rodriguez, B. W. Spur, Tetrahedron Lett. 2012, 53, 86-89; n) D. Chemin, G. Linstrumelle, Tetrahedron 1992, 48, 1943-1952; o) M. Treilhou, A. Fauve, H. Veschambre, J. R. Pougny, J. C. Promé, J. Org. Chem. 1992, 57, 3203-3208; p) K. Götz, J. C. Liermann, E. Thines, H. Anke, T. Opatz, Org. Biomol. Chem. 2010, 8, 2123-2130; q) J. E. Tungen, L. Gerstmann, A. Vik, R. De Matteis, R. A. Colas, J. Dalli, N. Chiang, C. N. Serhan, M. Kalesse, T. V. Hansen, Chem. Eur. J. 2019, 25, 1476-1480.
[21] Ł. Albrecht, H. Jiang, G. Dickmeiss, B. Gschwend, S. Grann Hansen, K. Anker Jorgensen, J. Am. Chem. Soc. 2010, 132, 9188-9196.
[22] M. Marigo, J. Franzén, T. B. Poulsen, W. Zhuang, K. A. Jorgensen, J. Am. Chem. Soc. 2005, 127, 6964-6965.
[23] R. L. Davis, K. L. Jensen, B. Gschwend, K. A. Jorgensen, Chem. Eur. J. 2014, 20, 64-67.
[24] W. Zhuang, M. Marigo, K. A. Jørgensen, Org. Biomol. Chem. 2005, 3, 3883-3885.
[25] See experimental details for the preparation of 22.
[26] See experimental details for the preparation of 5.
[27] J. Y. Sancéau, R. Maltais, D. Poirier, A. Marette, J. Org. Chem. 2019, 84, 495-505.
[28] Y. P. Sun, S. F. Oh, J. Uddin, R. Yang, K. Gotlinger, E. Campbell, S. P. Colgan, N. A. Petasis, C. N. Serhan, J. Biol. Chem. 2007, 282, 9323-9334.
[29] J. Li, M. M. Leong, A. Stewart, M. A. Rizzacasa, Beilstein J. Org. Chem. 2013, 9, 2762-2766.
[30] S. R. Woodcock, S. G. Wendell, F. J. Schopfer, B. A. Freeman, Tetrahedron Lett. 2018, 59, 3524-3527.
[31] Y. M. A. Mohamed, T. V. Hansen, Tetrahedron 2013, 69, 3872-3877.
[32] W. Boland, N. Schroer, C. Sieler, M. Feigel, Helv. Chim. Acta 1987, 70, 1025-1040.
[33] C. A. Brown, V. K. Ahuja, J. Chem. Soc. Chem. Commun. 1973, 553-554.
[34] C. A. Brown, V. K. Ahuja, J. Org. Chem. 1973, 38, 2226-2230.
[35] D. A. Rooke, E. M. Ferreira, Angew. Chem. Int. Ed. 2012, 124, 3279-3284.
[36]“Lipoxin B4 (LXB4, CAS Number: 98049-69-5)|Cayman Chemical,” can be found under https://www.caymanchem.com/product/90420/lipoxin-b4, n.d.
[37] See experimental for the structural assignment of LXB₄ based on 1D and 2D NMR.
[38] M. Tiberi, V. Chiurchiù, Front. Cell Neurosci. 2021, 15, 673549.
[39] S. Alqawlaq, I. Livne-Bar, D. Williams, J. D'Ercole, S. W. Leung, D. Chan, A. Tuccitto, A. Datti, J. L Wrana, A. H. Corbett, G. Schmitt-Ulms, J. M. Sivak, J. Biol. Chem. 2021, 296, 100118.
[40] S. Alqawlaq, J. G. Flanagan, J. M. Sivak, Exp. Eye Res. 2019, 183, 88-97.
[41] J. M. Sivak, Invest. Ophthalmol. Vis. Sci. 2013, 54, 871-880.
[42] N. Zhang, J. Wang, Y. Li, B. Jiang, Sci. Rep. 2021, 11, 13762.
[43]“Retrogenix Cell Microarray Technology|Charles River Laboraties,” https://www.criver.com/products-services/discovery-services/screening-and-profiling-assays/retrogenix-cell-microarray-technology?region=3601.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A process for synthesizing a compound of Formula I, 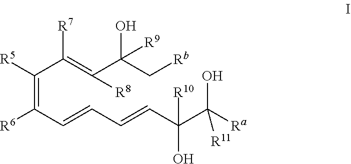
wherein
R⁵—R¹¹ are each independently H, ²H or ³H,
R^{a} is an optionally substituted alkyl or alkylene, and
R^{b} is an optionally substituted alkyl or alkylene,
said process comprising the steps of:
a) reacting a compound of Formula III with a vinyl halide of Formula IV to produce a compound of Formula V 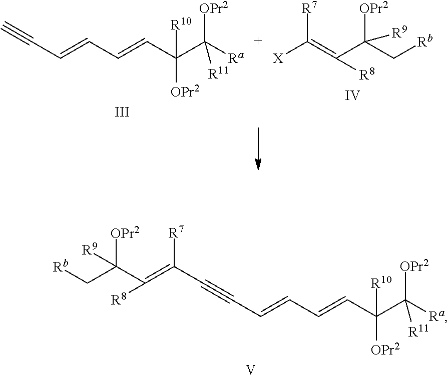
wherein each Pr² is independently an alcohol protecting group and X is a halide (e.g., I);
b) deprotecting the compound of Formula V to produce a compound of Formula Va 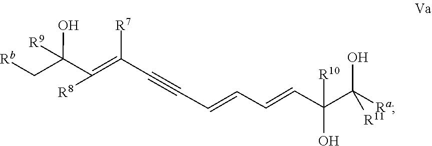
and
c) performing a selective semi-hydrogenation of the triple bond in the compound of Formula Va to form the compound of Formula I.

2. The process according to claim 1, wherein:
a) one or two of R⁵—R¹¹ are independently ²H or ³H; or
b) all of R⁵—R¹¹ are hydrogen.

3. The process according to claim 1, which is for synthesizing a compound of Formula I, 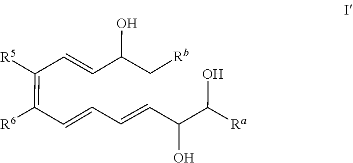
said process comprising the steps of:
a) reacting a compound of Formula III′ with a vinyl halide of Formula IV′ to produce a compound of Formula V′ 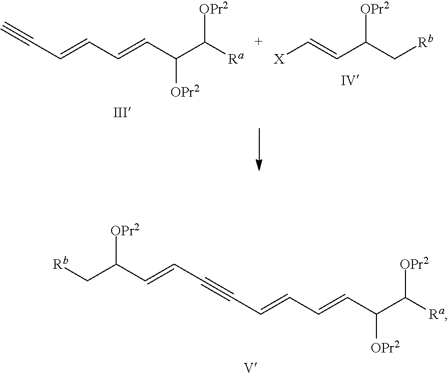
wherein each Pr² is independently an alcohol protecting group and X is a halide (e.g., I);
b) deprotecting the compound of Formula V′ to produce a compound of Formula Va′ 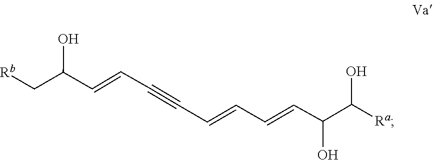
and
c) performing a selective semi-hydrogenation of the triple bond in the compound of Formula Va′ to form the compound of Formula I′.

4. The process of claim 1, wherein R^{a} is an optionally substituted C₁ to C₁₀ alkyl, such as pentyl and/or R^{b} is an optionally substituted C₁ to C₁₀ alkyl.

5. (canceled)

6. The process of claim 4, wherein R^{b} is a C₁ to C₁₀ alkyl substituted with a carboxylic acid, a carboxylate or an ester or salt thereof.

7. The process of claim 6, wherein R^{b} is substituted with an alkyl ester and the process additionally comprises step (d) of hydrolyzing the ester to produce the carboxylic acid or carboxylate, which is then optionally treated to form the ester or salt thereof.

8. The process of claim 7, wherein the compound of Formula I is a compound of Formula Ia: 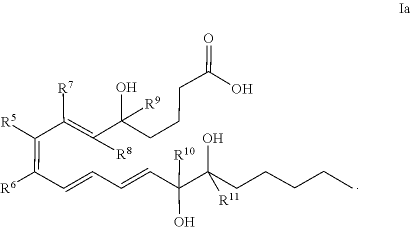
optionally wherein the compound of Formula Ia has the following stereochemistry:

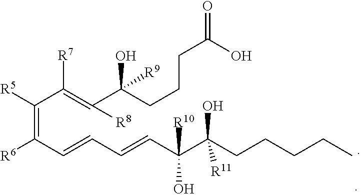

9. (canceled)

10. The process of claim 7, wherein the compound of Formula I is a compound of Formula Ia′: 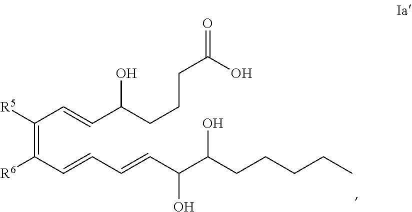
optionally wherein the compound of Formula Ia′ has the following stereochemistry:

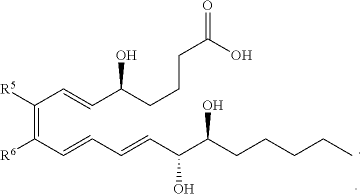

11. (canceled)

12. The process of claim 1, wherein each Pr² is independently an acetyl, acetonide, allyl, benzoyl, benzyl, b-methoxyethoxymethyl, methoxymethyl, methoxytrityl [(4-methoxyphenyl)diphenylmethyl], p-methoxybenzyl, p-methoxyphenyl, pivaloyl, tetrahydropyranyl, trityl, trimethylsilyl, triethylsilyl, tert-butyldimethylsilyl, tert-butyldiphenylsilyl, triisopropylsilyl, tri-iso-propylsilyloxymethyl, methyl, or ethoxyethyl protecting group.

13. The process of claim 1, wherein the compound of Formula I is lipoxin B4 or a deuterated or tritiated analogue of lipoxin B4.

14. (canceled)

15. A compound which has the structure of Formula Ia 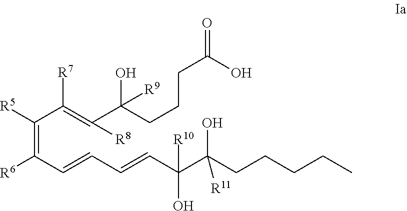
wherein R⁵—R¹¹ are each independently H, ²H or ³H, where at least one of R⁵—R¹¹ is ²H or ³H, or is a pharmaceutically acceptable salt, ester, hydrate, or solvate thereof.

16. The compound of claim 15, which has the structure of:
(a) Formula Ia′ 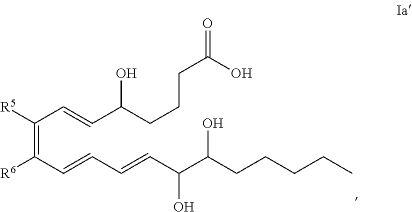
wherein R⁵ and R⁶ are the same and are deuterium or tritium, or
(b) a structure of Formula Ia″ 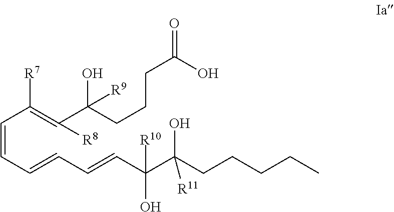
wherein one or two of R⁷—R¹¹ is ²H or ¹H, optionally wherein the compound has a structure that is: 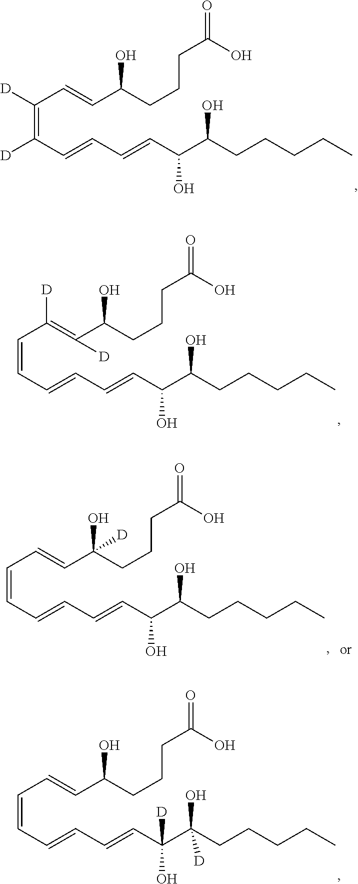
or
is a pharmaceutically acceptable salt, ester, hydrate, or solvate thereof.

17. The compound of claim 16, which is a compound of Formula Ia′, or a pharmaceutically acceptable salt, ester, hydrate or solvate thereof, wherein both R⁵ and R⁶ are deuterium.

18. (canceled)

19. The compound of claim 16, which is a compound of Formula Ia″, or a pharmaceutically acceptable salt, ester, hydrate or solvate thereof, wherein one or two of R⁷—R¹¹ are deuterium.

20.-24. (canceled)

25. A pharmaceutical composition comprising the compound according to claim 15, wherein at least one of R⁵—R¹¹ is deuterium, and a pharmaceutically acceptable diluent, excipient, carrier, or combination thereof.

26.-28. (canceled)

29. A method of providing neuroprotection, optionally retinal neuroprotection, or treating or preventing a disease or condition associated with neuroinflammation or neurodegeneration in a subject, comprising administering the compound of claim 15, wherein at least one of R⁵—R¹¹ is deuterium, to the subject.

30. The method according to claim 29, wherein the compound has the structure of:
(a) Formula Ia′ 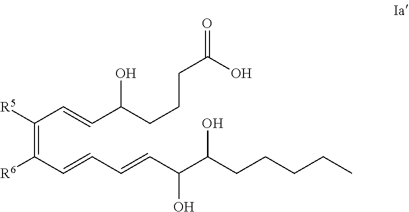
wherein both R⁵ and R⁶ are deuterium;
(b) Formula Ia″ 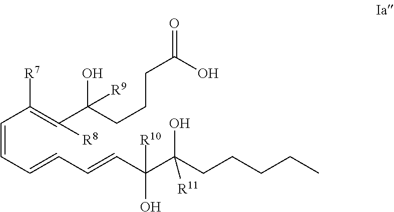
wherein one or two of R⁷—R¹¹ is ²H or ³H,
or is a pharmaceutically acceptable salt, ester, hydrate, or solvate thereof.

31. (canceled)

32. The method of claim 29, wherein the neuroprotection is for treatment of, and/or the neural disorder or condition is, central nervous system neurodegeneration and/or neural cell loss and the subject in need thereof is administered an amount of the compound such that neural degeneration and/or neuron cell loss is inhibited or prevented.

33. The method of claim 29, wherein the neural disorder or condition comprises hippocampal neuron, cortical neuron, optic neuron or retinal ganglion cell (RGC) neuron degeneration and/or cell loss.

34. The method of claim 29, wherein the neural disorder or condition comprises vision loss, an acute retinal or brain injury, optionally angle closure glaucoma, retinal vein occlusions, macular edema, ischemic and hemorrhagic stroke, and traumatic brain injury or a chronic neurodegenerative retinal or brain disorder such as glaucoma including all forms of primary open angle glaucoma, normal tension glaucoma, as well as retinal ischemias, diabetic retinopathy and diabetic macular edema, age related macular degeneration, retinitis pigmentosa, and Alzheimer's disease (retinal pathology), multiple sclerosis, as well as neurodegenerative brain diseases, such as Alzheimer's disease, Parkinson's disease and Amyotrophic Lateral Sclerosis (ALS).

35. The method of claim 29, wherein the neural disorder or condition comprises vision loss (or reduced vision), optionally wherein the disorder or condition is glaucoma.

36. The method of claim 29, wherein the compound is administered by parenteral, topical, intravenous, subcutaneous, intramuscular, intraorbital, ophthalmic, intraocular, intravitreal, intracameral, subtenon, subconjunctival, intraperitoneal, aerosol or oral administration.

37. The method of claim 29, wherein the compound is administered to an eye, optionally by a sustained delivery device, such as a contact lens, topical gel or ointment, polymer, or intraocular gel or sustained delivery device implant, polymer, or nanoparticles.

38. (canceled)

39. The method of claim 29, wherein the concentration of the compound administered is at least 0.2 nM or at least 50 nM and, optionally, less than 1 mM.

40. A compound of Formula II 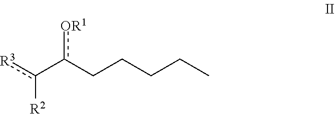
wherein
bond a is a single or double bond;
bond b is a single, double or triple bond;
when bond a is a single bond, R′ is H or an alcohol protecting group and when bond a is a double bond, R′ is absent;
when bond b is a single bond, R² is OR′, where R′ is H or an alcohol protecting group, and R³ is —CH═CH—CH═CH≡CH, —CH═CH—CH═O, —CH═CH—CH═CH≡C—Pr¹, —CH(OCH₃)₂, or 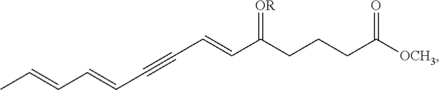
where R⁴ is H or an alcohol protecting group, or R² is O which forms an epoxide with the carbon to which R³ is attached and R³ is —CHO or —CH₂OR″, where R″ is an alcohol protecting group;
when bond b is a double bond R² is H and R³ is —CH₂OR″, wherein R″ is as defined above;
and when bond b is a triple bond R² is absent and R³ is —CH₂OR″, wherein R″ is as defined above.

41. The compound of claim 40, which is: [chemical structure]
